(19) United States
(12) Patent Application Publication
(10) Pub. No.: US 2006/0166901 A1
(43) Pub. Date: Jul. 27, 2006

(54) Compositions comprising O-acetylsalicyl derivatives of aminocarbohydrates and amino acids

(76) Inventors: Ruey J. Yu, Chalfont, PA (US); Eugene J. Van Scott, Abington, PA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/320,530

(22) Filed: Dec. 29, 2005

Related U.S. Application Data
(60) Provisional application No. 60/640,225, filed on Jan. 3, 2005.

(51) Int. Cl. A61K 31/7008; A61K 31/60

(52) U.S. Cl. 514/23; 514/165

(57) ABSTRACT

The embodiments described herein include a composition and method of treatment using compositions that include at least one O-acetylsalicyl derivative. The compositions and methods are useful in preventing and treating disorders and syndromes associated with anyone of the nervous, vascular, musculoskeletal, or cutaneous systems.

This application claims priority under 35 U.S.C. §119 to Provisional Patent Application No. 60/640,225 filed on Jan. 3, 2005, the disclosure of which is incorporated by reference herein in its entireties

### FIELD OF THE INVENTION

This application relates to compositions and methods of using compositions comprising an O-acetylsalicyl derivatives and a pharmaceutically acceptable excipient or vehicle for topical or systemic prevention or treatment to alleviate or improve pain, inflammation, disorders, symptoms or syndromes associated with the nervous, vascular, musculoskeletal or cutaneous systems.

### BACKGROUND

PCT Application No. PCT/US96/16534, filed Oct. 16,1996, entitled “Topical Compositions Containing N-Acetylcysteine and Odor Masking Materials,” describes topical compositions comprising from 0.01% to 50% of N-acetylcysteine or a derivative of N-acetylcysteine, from 0.01% to 0.5% of an odor masking material, and a topical carrier to improve the appearance of skin.

U.S. Pat. No. 6,159,485 entitled “N-Acetyl Aldosamines, N-Acetylamino Acids and Related N-Acetyl Compounds and Their Topical Use”, U.S. Pat. No. 6,524,593 B1 entitled “N-Acetyl Aldosamines and Related N-Acetyl Compounds, and Their Topical Use,” and U.S. Pat. No. 6,808,716 B2 entitled “N-Acetylamino Acids, Related N-Acetyl Compounds and Their Topical Use,” describe and claim the use of compositions comprising N-acetylamino acids and N-acetyl aldosamines for topical treatment of cosmetic conditions and dermatological disorders.

U.S. Pat. No. 6,824,786 B2 entitled “Compositions Comprising Phenyl-Glycine Derivatives” describes and claims the compositions and use of the compositions comprising phenyl-glycine derivatives for treating cosmetic conditions and dermatological disorders. The disclosures of each of the aforementioned United States patents are incorporated by reference herein in their entireties.

Aspirin is commonly used for temporary relief of pain and inflammation of arthritis and bursitis. The most common adverse reactions caused by oral aspirin is stomach irritation which leads to gastrointestinal bleeding in 70 percent of subjects taking repeated oral doses of aspirin. Corticosteroids such as prednisone and non-steroidal antiinflammatory drugs such as ibuprofen, naproxen, tolmetin and sulindac also may be used for temporary relief of arthritis. However, these drugs also can cause adverse side effects on long-term use. Rofecoxib (Vioxx) and celecoxib (Celebrex) are nonsteroidal anti-inflammatory, analgesic and antipyretic drugs for oral treatment of osteoarthritis, rheumatoid arthritis and analgesia including dysmenorrhea, dental pain and surgical pain. The mechanism of action by these two drugs is believed to be the suppression of prostaglandin synthesis through inhibition of cyclooxygenase-2. Recent studies have shown that patients who have taken rofecoxib or celecoxib had higher rates of heart attack (cardiac arrest) than the control group.

The description herein of disadvantages and problems associated with known compositions, and methods is in no way intended to limit the scope of the embodiments described in this document to their exclusion. Indeed, certain embodiments may include one or more known compositions, compounds, or methods without suffering from the so-noted disadvantages or problems.

### SUMMARY

It is a feature of an embodiment of the invention to provide a composition and method of preventing or alleviating symptoms or syndromes associated with the nervous, vascular, musculoskeletal, or cutaneous systems by topically or systemically administering to a subject in need thereof, a therapeutically effective amount of a composition comprising at least one O-acetylsalicyl derivative and a pharmaceutically acceptable excipient or vehicle. These and other features of various embodiments will become readily apparent to those skilled in the art upon review of the detailed description that follows.

### DETAILED DESCRIPTION

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth.

The inventors have discovered that compositions comprising an O-acetylsalicyl derivative, preferably an O-acetylsalicyl aminocarbohydrate or O-acetylsalicyl amino acid are beneficial or therapeutically effective for topical or systemic prevention or treatment to alleviate or improve symptoms or syndromes associated with nervous, vascular, musculoskeletal or cutaneous systems. The preferred compounds include N—(O-acetylsalicyl)-glucosamine, N—(O-acetylsalicyl)-mannosamine, N—(O-acetylsalicyl)-galactosamine, N—(O-acetylsalicyl)-glucamine N—(O-acetylsalicyl)-glycine ethyl ester, N—(O-acetylsalicyl)-tyrosine ethyl ester, N—(O-acetylsalicyl)-proline, N—(O-acetylsalicyl)-prolinamide, N—(O-acetylsalicyl)-proline ethyl ester, N—(O-acetylsalicyl)-glutamine, N—(O-acetylsalicyl)-glutamic acid, N—(O-acetylsalicyl)-glutamic acid diethyl ester, N—(O-acetylsalicyl)-arginine, N—(O-acetylsalicyl)-4-hydroxyphenylglycine ethyl ester; N,O-di(O-acetylsalicyl)-4-hydroxyphenylglycine ethyl ester, N—(O-acetylsalicyl)-3-phenylserine; N—(O-acetylsalicyl)-3-phenylserine ethyl ester; N—(O-acetylsalicyl)-lysine and N—(O-acetylsalicyl)-ornithine. These compounds are beneficial or therapeutically effective for topical or systemic prevention or treatment to alleviate or improve symptoms or syndromes associated with nervous, vascular, musculoskeletal or cutaneous systems. More specifically and preferably, these compounds are beneficial for oral administration to relieve pain and inflammation of the joints caused by arthritis or other disorders.

Because the aspirin (O-acetylsalicyl) moiety is chemically bound as an amide or ester linkage to an aminocarbohydrate or amino acid, the adverse reactions caused by oral aspirin such as gastrointestinal bleeding are eliminated or minimized.

The compositions described herein are therapeutically effective or beneficial by topical or systemic administration for disorders associated with nervous, vascular, musculoskeletal or cutaneous system. The systemic administration includes injection, infusion and oral administration, and the preferred route is by oral administration. These compounds are specifically beneficial for oral administration to relieve pain and inflammation of the joints caused by arthritis or other disorders.

The O-acetylsalicyl derivatives of the present embodiment preferably include are dimer or oligomer compounds formed from at least one O-acetylsalicyl radical and one aminocarbohydrate, amino acid or peptide through amide and/or ester bonds. The preferred O-acetylsalicyl derivatives of the present invention include N—(O-acetylsalicyl)-glucosamine, N—(O-acetylsalicyl)-mannosamine, N—(O-acetylsalicyl)-galactosamine, N—(O-acetylsalicyl)-glucamine, N—(O-acetylsalicyl)-glycine ethyl ester, N—(O-acetylsalicyl)-tyrosine ethyl ester, N—(O-acetylsalicyl)-proline, N—(O-acetylsalicyl)-prolinamide, N—(O-acetylsalicyl)-proline ethyl ester, N—(O-acetylsalicyl)-glutamine, N—(O-acetylsalicyl)-glutamic acid, N—(O-acetylsalicyl)-glutamic acid diethyl ester, N—(O-acetylsalicyl)-arginine, N—(O-acetylsalicyl)-4-hydroxyphenylglycine ethyl ester; N,O-di(O-acetylsalicyl)-4-hydroxyphenylglycine ethyl ester, N—(O-acetylsalicyl)-3-phenylserine; N—(O-acetylsalicyl)-3-phenylserine ethyl ester; N—(O-acetylsalicyl)-lysine and N—(O-acetylsalicyl)-ornithine.

The O-acetylsalicyl derivatives of the embodiments may be used in a method of improving, treating, ameliorating, alleviating, or reducing pains, inflammation, disorders, symptoms or syndromes associated with the nervous, vascular, musculoskeletal or cutaneous systems.

The symptoms and syndromes associated with the nervous system include, but are not limited to, (1) dementia and Alzheimer's disease: progressive loss of memory, shrinkage and atrophy of cerebral cortex, tangles of fibers in nerve cells, senile plaques of amyloid, decreased choline acetyltransferase enzyme, (2) carpal tunnel syndrome: weakness, pain, tingling, numbness, burning in palm and fingers, (3) encephalitis: inflammation of the brain, (4) headache: migraine, expansion of blood vessels pressing on nerves or constriction blocking blood supply, inflammation, muscle contraction to face, neck or scalp, (5) meningitis: infection of spinal fluid and meninges, (6) neuralgia: nerve pain, peripheral neuropathy, sciatica, shingles, trigeminal neuralgia, (7) Parkinson's disease: tremors in limbs, muscular rigidity, (8) amnesia: loss of memory and inability to form new memory, and others such as ataxia, Bell's palsy, epilepsy, multiple sclerosis, myasthenia gravis, narcolepsy, paralysis and rabies.

The vascular conditions, reactions and disorders include, but are not limited to, acanthosis nigricans, acrocyanosis, actinic cheilitis, actinic prurigo, dermatitis, dermatosis, dermographism, dyshidrosis, drug eruptions, eczema, erythema, erythema migrans, erythrocyanosis, erythromelalgia, familial hemorrhage, histamine reaction, inflammatory papular and pustular lesions, lichen planus, lupus erythematosus, mycosis fungoides, neurodermatitis, neuropeptide and neurovascular reactions, parapsoriasis, pemiosis(chilblains), photoallergy, photoreaction, photosensitivity, pityriasis rosea, pityriasis rubra pilaris, polymorphic light eruption, psoriasis, rhinophyma, rosacea, sclerosis, spider naevi, T-cell disorders, telangiectasia, urticaria and other vascular reactions.

The abnormalities of musculoskeletal system include, but are not limited to, (1) osteoporosis: reduction of calcium in bone leading to thin and susceptible to fracture, (2) osteoarthritis: inflammation of joint cartilage provoking swelling and pain, (3) rheumatoid arthritis: inflammation of synovium and destructions of cartilage, damage to heart, lungs, nerves and eyes, (4) ankylosing spondylitis: arthritis affecting sacroiliac joints and spine with inflammation and immovability, (5) bursitis: inflammation of bursa, (6) tendinitis: inflammation of tendon, (7) gout: recurrent acute arthritis from uric acid deposit, and (8) others such as general aches, dental pain, surgical pain, joint pains, backache, bunion and hernia.

The disorders of or abnormalities of cutaneous system include, but are not limited to, disturbed keratinization, pigmentation and immunity; inflammation; infections; disorders of oral, vaginal and anal mucosa; skin wound; and decreased physiological functions. The manifestations of cutaneous disorders include acne; age spots; blemished skin; blotches; cellulite; dermatoses; dandruff; dry skin; pruritus, eczema; ichthyosis; keratoses and hyperkeratoses; lentigines; melasmas; mottled skin; pseudofolliculitis barbae; photoaging and photodamage; psoriasis; skin lines; stretch marks; thinning of skin, nail plate and hair; wrinkles; xerosis; oral or gum disease; irritated, inflamed, unhealthy, damaged or abnormal mucosa, skin, hair, nail, anal or vaginal conditions; defective synthesis or repair of dermal components; abnormal or diminished synthesis of collagen, glycosaminoglycans, proteoglycans and elastin as well as diminished levels of such components in the dermis; uneven and rough surface of skin, nail and hair; loss or reduction of skin, nail and hair resiliency, elasticity and recoilability; lack of skin, nail and hair lubricants and luster; fragility and splitting of nail and hair; yellowing skin; reactive, irritating or telangiectatic skin; dull and older-looking skin, nail and hair; for skin bleach and lightening and wound healing.

The adverse reactions associated with oral aspirin include gastrointestinal bleeding, prolonged bleeding time, hemorrhage, peptic ulcer, salicylism and Reye's syndrome in children. Because the O-acetylsalicyl aminocarbohydrate, O-acetylsalicyl amino acid or related compound is a dimer or oligomer compound in which aspirin moiety is chemically linked by amide or ester bond, the adverse reactions as encountered by aspirin after oral administration are eliminated or minimized.

The O-acetylsalicyl aminocarbohydrates, O-acetylsalicyl amino acids and related compounds of the embodiments can be divided into six groups: (1) N—(O-acetylsalicyl)-aminocarbohydrates; (2) O—(O-acetylsalicyl)-aminocarbohydrates; (3) N,O-di(O-acetylsalicyl)-aminocarbohydrates; (4) N—(O-acetylsalicyl)-amino acids; (5) N—(O-acetylsalicyl)-peptides; and (6) related (O-acetylsalicyl)-derivatives.

### (1). N—(O-Acetylsalicyl)-aminocarbohydrates

An aminocarbohydrate can have multiple amino groups, but the preferred embodiment has only one amino group in the molecule, such as glucosamine and galactosamine. N—(O-Acetylsalicyl)-aminocarbohydrates are typically dimer compounds that are formed between O-acetylsalicyloyl group and the amino group of an aminocarbohydrate by an amide bond. For example, N—(O-acetylsalicyl)-glucosamine can be a dimer that can be formed by reacting O-acetylsalicyloyl chloride with glucosamine in aqueous solution. N—(O-Acetylsalicyl)-aminocarbohydrates also can be oligomer compounds that are formed between O-acetylsalicyloyl group and the amino group of an oligomer aminocarbohydrate containing 2 to 9 carbohydrate units. N—(O-Acetylsalicyl)-aminocarbohydrates can also contain more than one O-acetylsalicyloyl group by amide and/or ester bond. However, the preferred one is a dimer compound. The N—(O-acetylsalicyl)-aminocarbohydrates can be represented by the following generic structure:
R₁(CHOH)ₙ(CHNHR₂)R₃
where n is an integer, preferably 1-9; R₁ is selected from the group consisting of H, I, F, Cl, Br, CHO, CONH₂, COOR₄, an alkyl, alkoxyl, aralkyl or aryl group having 1 to 19 carbon atoms; R₂ is a radical group of O-acetylsalicyl, represented by CH₃COOC₆H₄CO; R₃ is selected from the group consisting of H, CHO, CH₂OH, CONH₂, and COOR₄; and R₄ is independently selected from the group consisting of H, an alkyl, aralkyl or aryl group having 1 to 9 carbon atoms; the H attached to any carbon atom can be substituted by I, F, Cl, Br, SH, CHO, CONH₂, NH₂, alkyl, alkoxyl, aralkyl or aryl group having 1 to 9 carbon atoms; the OH can be substituted by H, NH₂, NHCOCH₃, acetoxyl or other acyloxyl group having 2 to 9 carbon atoms.

N—(O-Acetylsalicyl)-aminocarbohydrates can be present as saturated or unsaturated, stereoisomeric or non-stereoisomeric, straight or branched chain or cyclic form, as a free acid, salt, partial salt, lactone, amide or ester form. A typical cyclic form of an N—(O-acetylsalicyl)-aminocarbohydrate is a five-member ring (furanose form) or six-member ring (pyranose form) of the aminocarbohydrate moiety. In the ring form, the structure is more complicated and is not readily represented by the above generic structure. In such case, the N—(O-Acetylsalicyl)-aminocarbohydrate will be represented by its chemical name.

The following are preferred representative N—(O-acetylsalicyl)-aminocarbohydrates—N—(O-acetylsalicyl)-glycerosamine, N—(O-acetylsalicyl)-erythrosamine, N—(O-acetylsalicyl)-threosamine, N—(O-acetylsalicyl)-ribosamine, N—(O-acetylsalicyl)-arabinosamine, N—(O-acetylsalicyl)-xylosamine, N—(O-acetylsalicyl)-lyxosamine, N—(O-acetylsalicyl)-allosamine, N—(O-acetylsalicyl)-allosamine, N—(O-acetylsalicyl)-glucosamine, N—(O-acetylsalicyl)-mannosamine, N—(O-acetylsalicyl)-gulosamine, N—(O-acetylsalicyl)-idosamine, N—(O-acetylsalicyl)-galactosamine, N—(O-acetylsalicyl)-talosamine, N—(O-acetylsalicyl)-alloheptosamine, N—(O-acetylsalicyl)-alloheptosamine, N—(O-acetylsalicyl)-glucoheptosamine, N—(O-acetylsalicyl)-mannoheptosamine, N—(O-acetylsalicyl)-guloheptosamine, N—(O-acetylsalicyl)-idoheptosamine, N—(O-acetylsalicyl)-galactoheptosamine, N—(O-acetylsalicyl)-taloheptosamine, N—(O-acetylsalicyl)-glyceraminic acid, N—(O-acetylsalicyl)-erythrosaminic acid, N—(O-acetylsalicyl)-threosaminic acid, N—(O-acetylsalicyl)-ribosaminic acid, N—(O-acetylsalicyl)-arabinosaminic acid, N—(O-acetylsalicyl)-xylosaminic acid, N—(O-acetylsalicyl)-lyxosaminic acid, N—(O-acetylsalicyl)-allosaminic acid, N—(O-acetylsalicyl)-altrosaminic acid, N—(O-acetylsalicyl)-glucosaminic acid, N—(O-acetylsalicyl)-mannosaminic acid, N—(O-acetylsalicyl)-gulosaminic acid, N—(O-acetylsalicyl)-idosaminic acid, N—(O-acetylsalicyl)-galactosaminic acid, N—(O-acetylsalicyl)-talosaminic acid, N—(O-acetylsalicyl)-alloheptosaminic acid, N—(O-acetylsalicyl)-altroheptosaminic acid, N—(O-acetylsalicyl)-glucoheptosaminic acid, N—(O-acetylsalicyl)-mannoheptosaminic acid, N—(O-acetylsalicyl)-guloheptosaminic acid, N—(O-acetylsalicyl)-idoheptosaminic acid, N—(O-acetylsalicyl)-galactoheptosaminic acid, N—(O-acetylsalicyl)-taloheptosaminic acid, N—(O-acetylsalicyl)lactosamine, N—(O-acetylsalicyl)muramic acid, N—(O-acetylsalicyl)neuramine, N—(O-acetylsalicyl)neuramin lactose, N—(O-acetylsalicyl)-neuraminic acid. N—(O-acetylsalicyl)-4-O-acetylneuraminic acid, N—(O-acetylsalicyl)-7-O-acetylneuraminic acid, N—(O-acetylsalicyl)-di-O-acetyineuraminic acid, N—(O-acetylsalicyl)-glycerosylamine, N—(O-acetylsalicyl)-erythrosylamine, N—(O-acetylsalicyl)-threosylamine, N—(O-acetylsalicyl)-ribosylamine, N—(O-acetylsalicyl)-arabinosylamine, N—(O-acetylsalicyl)-xylosylamine, N—(O-acetylsalicyl)-lyxosylamine, N—(O-acetylsalicyl)-allosylamine, N—(O-acetylsalicyl)-altrosylamine, N—(O-acetylsalicyl)-glucosylamine [same as N—(O-acetylsalicyl)-glucamine], N—(O-acetylsalicyl)-mannosylamine, N—(O-acetylsalicyl)-gulosylamine, N—(O-acetylsalicyl)-idosylamine, N—(O-acetylsalicyl)-galactosylamine, N—(O-acetylsalicyl)-talosylamine, N—(O-acetylsalicyl)-alloheptosylamine, N—(O-acetylsalicyl)-altroheptosylamine, N—(O-acetylsalicyl)-glucoheptosylamine, N—(O-acetylsalicyl)-mannoheptosylamine, N—(O-acetylsalicyl)-guloheptosylamine, N—(O-acetylsalicyl)-idoheptosylamine, N—(O-acetylsalicyl)-galactoheptosylamine, N—(O-acetylsalicyl)-taloheptosylamine, wherein the suffix “-sylamine” represents the same as that of “-camine,” and mixtures and combinations thereof.

### (2) O—(O-Acetylsalicyl)-aminocarbohydrates

An aminocarbohydrate such as glucosamine usually has one amino group and four hydroxyl groups, and the O-acetylsalicyl radical is attached to any one of the four hydroxyl groups. The O—(O-Acetylsalicyl)-aminocarbohydrate can be a dimer or oligomer, depending on the number of ester bonds formed between O-acetylsalicyloyl radical and the hydroxyl group(s) of the aminocarbohydrate. For example, when one O-acetylsalicyloyl radical is attached to one hydroxyl group of glucosamine, the O—(O-acetylsalicyl)-glucosamine thus formed is a dimer compound. When two O-acetylsalicyloyl radicals are attached to two hydroxyl groups of glucosamine, the di-O—(O-acetylsalicyl)-glucosamine thus formed is a trimer compound. In the same manner, tri-O—(O-acetylsalicyl)-glucosamine (tetramer) and tetra-O—(O-acetylsalicyl)-glucosamine (pentamer) can be formed. The preferred O—(O-acetylsalicyl)-aminocarbohydrates is a dimer and has only one ester bond. The ester bond can be formed at any one of the hydroxyl groups, although the preferred one is at the anomeric carbon of the aminocarbohydrate. For example, 1-O—(O-acetylsalicyl)-glucosamine is formed by an ester bond at position one of the glucosamine when the latter is in a pyranose or furanose form. The O—(O-acetylsalicyl)-aminocarbohydrates of dimer compounds can be represented by the following generic structure:
R₁(CHOR₂)ₙ(CHNH₂)R₃
where n is an integer, preferably 1-9; R₁ is selected from the group consisting of H, I, F, Cl, Br, CHO, CONH₂, COOR₄, an alkyl, alkoxyl, aralkyl or aryl group having 1 to 19 carbon atoms; R₂ is H or at least one R₂ is a radical group of O-acetylsalicyl, represented by CH₃COOC₆H₄CO; R₃ is selected from the group consisting of H, CHO, CH₂OH, CONH₂, and COOR₄; and R₄ is independently selected from the group consisting of H, an alkyl, aralkyl or aryl group having 1 to 9 carbon atoms; the H attached to any carbon atom can be substituted by I, F, Cl, Br, SH, CHO, CONH₂, NH₂, alkyl, alkoxyl, aralkyl or aryl group having 1 to 9 carbon atoms; the OH can be substituted by H, NH₂, NHCOCH₃, acetoxyl or other acyloxyl group having 2 to 9 carbon atoms.

O—(O-Acetylsalicyl)-aminocarbohydrates can be present as saturated or unsaturated, stereoisomeric or non-stereoisomeric, straight or branched chain or cyclic form, as a free acid, salt, partial salt, lactone, amide or ester form. A typical cyclic form of an O—(O-acetylsalicyl)-aminocarbohydrates is a five member ring (furanose form) or six member ring (pyranose form) of the aminocarbohydrate moiety. In the ring form, the structure is more complicated and is not readily represented by the above generic structure. In such case, the O—(O-Acetylsalicyl)-aminocarbohydrate will be represented by its chemical name.

The following are representative O—(O-acetylsalicyl)-aminocarbohydrates-O—(O-acetylsalicyl)-glycerosamine, O—(O-acetylsalicyl)-erythrosamine, O—(O-acetylsalicyl)-threosamine, O—(O-acetylsalicyl)-ribosamine, O—(O-acetylsalicyl)-arabinosamine, O—(O-acetylsalicyl)-xylosamine, O—(O-acetylsalicyl)-lyxosamine, O—(O-acetylsalicyl)-allosamine, O—(O-acetylsalicyl)-altrosamine, O—(O-acetylsalicyl)-glucosamine, O—(O-acetylsalicyl)-mannosamine, O—(O-acetylsalicyl)-gulosamine, O—(O-acetylsalicyl)-idosamine, O—(O-acetylsalicyl)-galactosamine, O—(O-acetylsalicyl)-talosamine, O—(O-acetylsalicyl)-alloheptosamine, O—(O-acetylsalicyl)-altroheptosamine, O—(O-acetylsalicyl)-glucoheptosamine, O—(O-acetylsalicyl)-mannoheptosamine, O—(O-acetylsalicyl)-guloheptosamine, O—(O-acetylsalicyl)-idoheptosamine, O—(O-acetylsalicyl)-galactoheptosamine, O—(O-acetylsalicyl)-taloheptosamine, O—(O-acetylsalicyl)-glyceraminic acid, O—(O-acetylsalicyl)-erythrosaminic acid, O—(O-acetylsalicyl)-threosaminic acid, O—(O-acetylsalicyl)-ribosaminic acid, O—(O-acetylsalicyl)-arabinosaminic acid, O—(O-acetylsalicyl)-xylosaminic acid, O—(O-acetylsalicyl)-lyxosaminic acid, O—(O-acetylsalicyl)-allosaminic acid, O—(O-acetylsalicyl)-altrosaminic acid, O—(O-acetylsalicyl)-glucosaminic acid, O—(O-acetylsalicyl)-mannosaminic acid, O—(O-acetylsalicyl)-gulosaminic acid, O—(O-acetylsalicyl)-idosaminic acid, O—(O-acetylsalicyl)-galactosaminic acid, O—(O-acetylsalicyl)-talosaminic acid, O—(O-acetylsalicyl)-alloheptosaminic acid, O—(O-acetylsalicyl)-altroheptosaminic acid, O—(O-acetylsalicyl)-glucoheptosaminic acid, O—(O-acetylsalicyl)-mannoheptosaminic acid, O—(O-acetylsalicyl)-guloheptosaminic acid, O—(O-acetylsalicyl)-idoheptosaminic acid, O—(O-acetylsalicyl)-galactoheptosaminic acid, O—(O-acetylsalicyl)-taloheptosaminic acid, O—(O-acetylsalicyl)lactosamine, O—(O-acetylsalicyl)muramic acid, O—(O-acetylsalicyl)neuramine, O—(O-acetylsalicyl)neuramin lactose, O—(O-acetylsalicyl)-neuraminic acid. O—(O-acetylsalicyl)-4-O-acetylneuraminic acid, O—(O-acetylsalicyl)-7-O-acetylneuraminic acid, O—(O-acetylsalicyl)-di-O-acetylneuraminic acid, O—(O-acetylsalicyl)-glycerosylamine, O—(O-acetylsalicyl)-erythrosylamine, O—(O-acetylsalicyl)-threosylamine, O—(O-acetylsalicyl)-ribosylamine, O—(O-acetylsalicyl)-arabinosylamine, O—(O-acetylsalicyl)-xylosylamine, O—(O-acetylsalicyl)-lyxosylamine, O—(O-acetylsalicyl)-allosylamine, O—(O-acetylsalicyl)-altrosylamine, O—(O-acetylsalicyl)-glucosylamine [same as O—(O-acetylsalicyl)-glucamine], O—(O-acetylsalicyl)-mannosylamine, O—(O-acetylsalicyl)-gulosylamine, O—(O-acetylsalicyl)-idosylamine, O—(O-acetylsalicyl)-galactosylamine, O—(O-acetylsalicyl)-talosylamine, O—(O-acetylsalicyl)-alloheptosylamine, O—(O-acetylsalicyl)-altroheptosylamine, O—(O-acetylsalicyl)-glucoheptosylamine, O—(O-acetylsalicyl)-mannoheptosylamine, O—(O-acetylsalicyl)-guloheptosylamine, O—(O-acetylsalicyl)-idoheptosylamine, O—(O-acetylsalicyl)-galactoheptosylamine, O—(O-acetylsalicyl)-taloheptosylamine, wherein the suffix “-sylamine” represents the same as that of “-camine,” and mixtures and combinations thereof.

### (3) N,O-Di(O-acetylsalicyl)-aminocarbohydrates

An aminocarbohydrate such as glucosamine usually has one amino group and four hydroxyl groups. One O-acetylsalicyl radical can be attached to the amino group and a second radical can be attached to any one of the four hydroxyl groups. The N,O-di(O-acetylsalicyl)-aminocarbohydrate thus formed is a trimer compound that is formed between two O-acetylsalicyl radicals and one aminocarbohydrate by one amide and one ester bond. For example, N,O-di(O-acetylsalicyl)-glucosamine is a trimer compound that consists of two O-acetylsalicyl radicals and one glucosamine formed by one amide and one ester bond. When one O-acetylsalicyl radical is attached to the amino group and two O-acetylsalicyl radicals are attached to two hydroxyl groups of the aminocarbohydrate, the N,O,O-tri(O-acetylsalicyl)-aminocarbohydrate thus formed is a tetramer. In the same manner; N,O,O,O-tetra(O-acetylsalicyl)-aminocarbohydrate (pentamer); N,O,O,O,O-penta(O-acetylsalicyl)-aminocarbohydrate (hexamer) and N,O,O,O,O,O-hexa(O-acetylsalicyl)-aminocarbohydrate (heptamer) can be formed. The preferred N,O-di(O-acetylsalicyl)-aminocarbohydrates can be represented by the following generic structure:
R₁(CHOR₂)ₙ(CHNHCOC₆H₄OCOCH₃)R₃
where n is an integer, preferably 1-9; R₁ is selected from the group consisting of H, I, F, Cl, Br, CHO, CONH₂, COOR₄, an alkyl, alkoxyl, aralkyl or aryl group having 1 to 19 carbon atoms; R₂ is H or at least one R₂ is a radical group of O-acetylsalicyl, represented by CH₃COOC₆H₄CO; R₃ is selected from the group consisting of H, CHO, CH₂OH, CONH₂, and COOR₄; and R₄ is independently selected from the group consisting of H, an alkyl, aralkyl or aryl group having 1 to 9 carbon atoms; the H attached to any carbon atom can be substituted by I, F, Cl, Br, SH, CHO, CONH₂, NH₂, alkyl, alkoxyl, aralkyl or aryl group having 1 to 9 carbon atoms.

The N,O-di(O-Acetylsalicyl)-aminocarbohydrates can be present as saturated or unsaturated, stereoisomeric or non-stereoisomeric, straight or branched chain or cyclic form, as a free acid, salt, partial salt, lactone, amide or ester form. A typical cyclic form of an N,O-di(O-acetylsalicyl)-aminocarbohydrate is a five member ring (furanose form) or six member ring (pyranose form) of the aminocarbohydrate moiety. In the ring form, the structure is more complicated and is not readily represented by the above generic structure. In such case, the N,O-di(O-Acetylsalicyl)-aminocarbohydrate will be represented by its chemical name.

The following are representative N,O-di(O-acetylsalicyl)-aminocarbohydrates—N,O-di(O-acetylsalicyl)-glycerosamine; N,O-di(O-acetylsalicyl)-erythrosamine; N,O-di(O-acetylsalicyl)-threosamine; N,O-di(O-acetylsalicyl)-ribosamine; N,O-di(O-acetylsalicyl)-arabinosamine; N,O-di(O-acetylsalicyl)-xylosamine; N,O-di(O-acetylsalicyl)-lyxosamine; N,O-di(O-acetylsalicyl)-allosamine; N,O-di(O-acetylsalicyl)-altrosamine; N,O-di(O-acetylsalicyl)-glucosamine; N,O-di(O-acetylsalicyl)-mannosamine; N,O-di(O-acetylsalicyl)-gulosamine; N,O-di(O-acetylsalicyl)-idosamine; N,O-di(O-acetylsalicyl)-galactosamine; N,O-di(O-acetylsalicyl)-talosamine; N,O-di(O-acetylsalicyl)-alloheptosamine; N,O-di(O-acetylsalicyl)-altroheptosamine; N,O-di(O-acetylsalicyl)-glucoheptosamine; N,O-di(O-acetylsalicyl)-mannoheptosamine; N,O-di(O-acetylsalicyl)-guloheptosamine; N,O-di(O-acetylsalicyl)-idoheptosamine; N,O-di(O-acetylsalicyl)-galactoheptosamine; N,O-di(O-acetylsalicyl)-taloheptosamine; N,O-di(O-acetylsalicyl)-glyceraminic acid; N,O-di(O-acetylsalicyl)-erythrosaminic acid; N,O-di(O-acetylsalicyl)-threosaminic acid; N,O-di(O-acetylsalicyl)-ribosaminic acid; N,O-di(O-acetylsalicyl)-arabinosaminic acid; N,O-di(O-acetylsalicyl)-xylosaminic acid; N,O-di(O-acetylsalicyl)-lyxosaminic acid; N,O-di(O-acetylsalicyl)-allosaminic acid; N,O-di(O-acetylsalicyl)-altrosaminic acid; N,O-di(O-acetylsalicyl)-glucosaminic acid; N,O-di(O-acetylsalicyl)-mannosaminic acid; N,O-di(O-acetylsalicyl)-gulosaminic acid; N,O-di(O-acetylsalicyl)-idosaminic acid; N,O-di(O-acetylsalicyl)-galactosaminic acid; N,O-di(O-acetylsalicyl)-talosaminic acid; N,O-di(O-acetylsalicyl)-alloheptosaminic acid; N,O-di(O-acetylsalicyl)-altroheptosaminic acid; N,O-di(O-acetylsalicyl)-glucoheptosaminic acid; N,O-di(O-acetylsalicyl)-mannoheptosaminic acid; N,O-di(O-acetylsalicyl)-guloheptosaminic acid; N,O-di(O-acetylsalicyl)-idoheptosaminic acid; N,O-di(O-acetylsalicyl)-galactoheptosaminic acid; N,O-di(O-acetylsalicyl)-taloheptosaminic acid; N,O-di(O-acetylsalicyl)lactosamine; N,O-di(O-acetylsalicyl)muramic acid; N,O-di(O-acetylsalicyl)neuramine; N,O-di(O-acetylsalicyl)neuramin lactose; N,O-di(O-acetylsalicyl)-neuraminic acid; N,O-di(O-acetylsalicyl)-4-O-aeacetylneuraminic acid; N,O-di(O-acetylsalicyl)-7-O-acetylneuraminic acid; N,O-di(O-acetylsalicyl)-di-O-acetylneuraminic acid, N,O-di(O-acetylsalicyl)-glycerosylamine; N,O-di(O-acetylsalicyl)-erythrosylamine; N,O-di(O-acetylsalicyl)-threosylamine; N,O-di(O-acetylsalicyl)-ribosylamine; N,O-di(O-acetylsalicyl)-arabinosylamine; N,O-di(O-acetylsalicyl)-xylosylamine; N,O-di(O-acetylsalicyl)-lyxosylamine; N,O-di(O-acetylsalicyl)-allosylamine; N,O-di(O-acetylsalicyl)-altrosylamine; N,O-di(O-acetylsalicyl)-glucosylamine [same as N,O-di(O-acetylsalicyl)-glucamine]; N,O-di(O-acetylsalicyl)-mannosylamine; N,O-di(O-acetylsalicyl)-gulosylamine; N,O-di(O-acetylsalicyl)-idosylamine; N,O-di(O-acetylsalicyl)-galactosylamine; N,O-di(O-acetylsalicyl)-talosylamine; N,O-di(O-acetylsalicyl)-alloheptosylamine; N,O-di(O-acetylsalicyl)-altroheptosylamine; N,O-di(O-acetylsalicyl)-glucoheptosylamine; N,O-di(O-acetylsalicyl)-mannoheptosylamine; N,O-di(O-acetylsalicyl)-guloheptosylamine; N,O-di(O-acetylsalicyl)-idoheptosylamine; N,O-di(O-acetylsalicyl)-galactoheptosylamine; and N,O-di(O-acetylsalicyl)-taloheptosylamine, wherein the suffix “-sylamine” represents the same as that of “-camine,” and mixtures and combinations thereof.

### (4) N—(O-acetylsalicyl)-amino acids

The N—(O-acetylsalicyl)-amino acids preferably are dimer or other oligomer compounds chemically linked by an amide bond(s) and preferably are formed between at least one O-acetylsalicyloyl radical and the amino group(s) of an amino acid. The N—(O-acetylsalicyl)-amino acids can be represented by the following generic structure:
R₁CHNHR₂(CH₂)ₙCOR₃
where R₁ is H, an alkyl, aralkyl or aryl group having 1 to 14 carbon atoms, and R₁ can also carry OH, SH, SCH₃, NH₂, NHR₂, CONH₂, NHCONH₂, NHC(═NH)NH₂, NHC(═NR₂)NH₂, imidazole, pyrrolidine or other heterocyclic group; R₂ is O-acetylsalicyl radical, represented by CH₃COOC₆H₄CO; n is an integer from 0 to 5; R₃ is NH₂, OR₄; R₄ is H, an alkyl, aralkyl or aryl group having 1 to 14 carbon atoms; and the H attached to any carbon atom can be substituted by I, F, Cl, Br, OH or alkoxy group having 1 to 9 carbons.

Among commonly known amino acids, proline cannot be represented by the above generic structure because the alpha amino group is part of the heterocyclic pyrrolidine ring. Therefore, N—(O-acetylsalicyl)-proline is represented by chemical name only. The N—(O-acetylsalicyl)-amino acids can be present as stereoisomers such as D, L or DL, such as N—(O-acetylsalicyl)-L-tyrosine, or non-stereoisomers such as N—(O-acetylsalicyl)-glycine, as saturated or unsaturated, straight or branched chain, or cyclic form, as a free acid, salt, partial salt, lactone, amide or ester form. N—(O-Acetylsalicyl)amino acids will be described only by their chemical names if the structures are too complicated to be covered by the above generic structure.

The following are representative N—(O-acetylsalicyl)amino acids—N—(O-acetylsalicyl)-alanine, N—(O-acetylsalicyl)-arginine; N,N′-di(O-acetylsalicyl)-arginine, N—(O-acetylsalicyl)-asparagine, N—(O-acetylsalicyl)-aspartic acid, N—(O-acetylsalicyl)-cysteine, N—(O-acetylsalicyl)-glycine, N—(O-acetylsalicyl)-glutamic acid, N—(O-acetylsalicyl)-glutamine, N—(O-acetylsalicyl)-histidine; N,N-di(O-acetylsalicyl)-histidine, N—(O-acetylsalicyl)-isoleucine, N—(O-acetylsalicyl)-leucine, N—(O-acetylsalicyl)-lysine; N,N-di(O-acetylsalicyl)-lysine, N—(O-acetylsalicyl)-methionine, N—(O-acetylsalicyl)-phenylalanine, N—(O-acetylsalicyl)-proline, N—(O-acetylsalicyl)-serine, N—(O-acetylsalicyl)-threonine, N—(O-acetylsalicyl)-tryptophan; N,N′-di(O-acetylsalicyl)-tryptophan, N—(O-acetylsalicyl)-tyrosine, N—(O-acetylsalicyl)-valine, N—(O-acetylsalicyl)-β-alanine, N—(O-acetylsalicyl)-γ-aminobutanoic acid, N—(O-acetylsalicyl)-β-aminoisobutanoic acid, N—(O-acetylsalicyl)-citrulline, N—(O-acetylsalicyl)-dopa(3,4-dihydroxyphenylalanine), N—(O-acetylsalicyl)-homocysteine, N—(O-acetylsalicyl)-homoserine, N—(O-acetylsalicyl)-ornithine; N,N-di(O-acetylsalicyl)-ornithine N—(O-acetylsalicyl)-phenylglycine and N—(O-acetylsalicyl)-4-hydroxyphenylglycine, and mixtures and combinations thereof.

### (5) N—(O-acetylsalicyl)-peptides

The N—(O-acetylsalicyl)-peptides are preferably trimer or other oligomer compounds chemically linked by amide bond(s) and are formed between an O-acetylsalicyloyl radical and the amino group of a dipeptide or other oligopeptide. The oligopeptide usually contains 2 to 10 amino acid units. However, the preferred N—(O-acetylsalicyl)-peptides are trimer compounds formed between one O-acetylsalicyloyl radical and a dipeptide through an amide bond. The N—(O-acetylsalicyl)-dipeptides can be represented by the following generic structure:
R₁CHNHR₂CO NHCHR₁COR₃
where R₁ is independently H, an alkyl, aralkyl or aryl group having 1 to 14 carbon-atoms, and can carry OH, SH, SCH₃, NH₂, CONH₂, NHCONH₂, NHC(═NH)NH₂, imidazole, pyrrolidine or other heterocyclic group; R₂ is O-acetylsalicyl radical, represented by CH₃COOC₆H₄CO; R₃ is NH₂, OR₄; R₄ is H, an alkyl, aralkyl or aryl group having 1 to 14 carbon atoms; and the H attached to any carbon atom can be substituted by I, F, Cl, Br, OH or alkoxy group having 1 to 9 carbons.

The N—(O-acetylsalicyl)-peptides can be present as stereoisomers such as D,L,or DL, such as N—(O-acetylsalicyl)-L-Tyr-L-Tyr or non-stereoisomers such as N—(O-acetylsalicyl)-Gly-Gly. Among commonly known amino acid units, proline cannot be represented by the above generic structure because the alpha amino group is part of the heterocyclic pyrrolidine ring. Therefore, N—(O-acetylsalicyl)-proline peptides are represented by names only. N—(O-Acetylsalicyl)-peptides can be present as saturated or unsaturated, straight or branched chain, or cyclic form, as a free acid, salt, partial salt, lactone, amide or ester form. Many N—(O-acetylsalicyl)-oligopeptides are represented by their chemical names only because they cannot be represented by the above generic structure.

The following are representative N—(O-acetylsalicyl)-dipeptides—N—(O-acetylsalicyl)-kyotorphin (Tyr-Arg), N—(O-acetylsalicyl)-anserine (β-Ala-1-N-Me-His), N—(O-acetylsalicyl)-β-Ala-Lys, N—(O-acetylsalicyl)-Asp-Glu, N—(O-acetylsalicyl)-carnosine (β-Ala-His), N—(O-acetylsalicyl)-Gly-Gln, N—(O-acetylsalicyl)-γ-D-Glu-Gly, N—(O-acetylsalicyl)-γ-Glu-Val, N—(O-acetylsalicyl)-γ-Glu-Ile, N—(O-acetylsalicyl)-γ-Glu-Leu, N—(O-acetylsalicyl)-γ-Glu-Met, N—(O-acetylsalicyl)-γ-Glu-Phe, N—(O-acetylsalicyl)-γ-Glu-Ala, N—(O-acetylsalicyl)-γ-Glu-Tyr, N—(O-acetylsalicyl)-γ-Glu-Phe, N—(O-acetylsalicyl)-Leu-Leu, N—(O-acetylsalicyl)-ophidine (β-Ala-2-Me-His), N—(O-acetylsalicyl)-Met-Phe, N—(O-acetylsalicyl)-Ser-Tyr-NH₂, N—(O-acetylsalicyl)-Tyr-Pro-NH₂, and mixtures and combinations thereof.

The following are representative N—(O-acetylsalicyl)-tripeptides—N—(O-acetylsalicyl)-bursin (Lys-H is-Gly-N H₂), N—(O-acetylsalicyl)-D-Phe-Phe-Gly; N—(O-acetylsalicyl)-diprotin A (Ile-Pro-Ile), N—(O-acetylsalicyl)-glutathione (γ-Glu-Cys-Gly), N—(O-acetylsalicyl)-pGlu-Glu-Pro-NH₂, N—(O-acetylsalicyl)-Lys-Trp-Lys, N—(O-acetylsalicyl)-Lys-Tyr-Lys, N—(O-acetylsalicyl)-ophthalmic acid, N—(O-acetylsalicyl)-norophthalmic acid (γ-Glu-Ala-Gly), N—(O-acetylsalicyl)-homoglutathione (bis-γ-Glu-Cys-bis-β-Ala), N—(O-acetylsalicyl)-Met-Leu-Phe, N—(O-acetylsalicyl)-Tyr-Gly-Gly, N—(O-acetylsalicyl)-Pro-Leu-Gly-NH₂, and mixtures and combinations thereof.

The following are further representative N—(O-acetylsalicyl)-oligopeptides—N—(O-acetylsalicyl)-Lys-Lys-Gly-Glu, N—(O-acetylsalicyl)-Arg-Pro-Lys-Pro, N—(O-acetylsalicyl)-Gly-Gly-Phe-Leu, N—(O-acetylsalicyl)-Arg-Gly-Asp-Ser, N—(O-acetylsalicyl)-Arg-Gly-Glu-Ser, N—(O-acetylsalicyl)-Met-Leu-Phe-Phe, N—(O-acetylsalicyl)-Val-Ala-Pro-Gly, N—(O-acetylsalicyl)-Val-Gly-Ser-Glu, N—(O-acetylsalicyl)-Ala-Pro-Gly-Trp-N H₂, N—(O-acetylsalicyl)-Gly-Gly-Phe-Met-NH₂, N—(O-acetylsalicyl)-endorphin 1 (Tyr-Pro-Trp-Phe-NH₂), N-(O-acetylsalicyl)-endorphin 2 (Tyr-Pro-Phe-Phe-N H₂), N—(O-acetylsalicyl)-morphiceptin (Tyr-Pro-Phe-Pro-NH₂), N—(O-acetylsalicyl)-exorphin C (Tyr-Pro-Ile-Ser-Leu), N—(O-acetylsalicyl)-pyroGlu-Glu-Asp-Ser-Gly, N—(O-acetylsalicyl)-Tyr-Gly-Gly-Phe-Leu, N—(O-acetylsalicyl)-Tyr-Gly-Gly-Phe-Met, N—(O-acetylsalicyl)-pyroGlu-Leu-Tyr-Glu-Asn-Lys, N—(O-acetylsalicyl)-Arg-Arg-Pro-Tyr-Ile-Leu, N—(O-acetylsalicyl)-Val-Gly-Val-Ala-Pro-Gly, N-(O-acetylsalicyl)-Tyr-Pro-Ile-Ser-Leu, N—(O-acetylsalicyl)-Arg-Lys-Arg-Ala-Arg-Lys-Glu, N—(O-acetylsalicyl)-kemptide (Leu-Arg-Arg-Ala-Ser-Leu-Gly), N—(O-acetylsalicyl)-Tyr-Gly-Gly-Phe-Leu-Arg-Arg, N—(O-acetylsalicyl)-Met-Glu-His-Phe-Arg-Trp-Gly, N—(O-acetylsalicyl)-Arg-Val-Tyr-Ile-His-Pro-Phe, N—(O-acetylsalicyl)-β-casomorphin (Tyr-Pro-Phe-Val-Glu-Pro-Ile), N—(O-acetylsalicyl)-Asp-Arg-Val-Tyr-Ile-His-Pro-Phe, N—(O-acetylsalicyl)-Met-Gln-Met-Lys-Lys-Val-Leu-Asp-Ser, N—(O-acetylsalicyl)-Arg-Pro-Pro-Gly-Phe-Ser-Pro-Phe-Arg, N—(O-acetylsalicyl)-Ile-Ala-Arg-Arg-His-Pro-Tyr-Phe-Leu, N—(O-acetylsalicyl)-Cys-Tyr-Ile-Gln-Asn-Cys-Pro-Leu-Gly-NH₂, N—(O-acetylsalicyl)-Cys-Tyr-Phe-Gln-Asn-Cys-Pro-Arg-Gly-NH₂, N—(O-acetylsalicyl)-Cys-Tyr-Ile-Gln-Asn-Cys-Pro-Arg-Gly-NH₂, and mixtures and combinations thereof.

### (6) Related (O-acetylsalicyl)-derivatives

The related derivatives include: O—(O-acetylsalicyl)-amino acids; N,O-di(O-acetylsalicyl)-amino acids; N,O,O-tri(O-acetylsalicyl)-amino acids; S-(O-acetylsalicyl)-amino acids; N,S-di (O-acetylsalicyl)-amino acids; O,S-di(O-acetylsalicyl)-amino acids, and preferably are derived from those amino acids with hydroxyl or thiol group(s). Other (O-acetylsalicyl)-derivatives include: N—(O-acetylsalicyl)-amino-oligosaccharides; N—(O-acetylsalicyl)-oligo-aminosaccharides; N,O-di(O-acetylsalicyl)-amino-oligosaccharides; and N,O-di(O-acetylsalicyl)-oligo-aminosaccharides. In amino-oligosaccharides and oligo-aminosaccharides, O-acetylsalicyl radicals can be attached to the amino groups and/or hydroxyl groups. Examples of the above amino acids include serine, homoserine, 3-phenylserine, threonine, tyrosine, 4-hydroxyphenylglycine, cysteine, homocysteine and dopa.

The following are representative (O-acetylsalicyl)amino acid derivatives—S—(O-acetylsalicyl)-cysteine; N,S-di(O-acetylsalicyl)-cysteine; S—(O-acetylsalicyl)-homocysteine; N,S-di(O-acetylsalicyl)-homocysteine; O—(O-acetylsalicyl)-4-hydroxyphenylglycine; N,O-di(O-acetylsalicyl)-4-hydroxyphenylglycine; O—(O-acetylsalicyl)-serine; N,O-di(O-acetylsalicyl)-serine; O—(O-acetylsalicyl)-homoserine; N,O-di(O-acetylsalicyl)-homoserine; O—(O-acetylsalicyl)-3-phenylserine; N,O-di(O-acetylsalicyl)-3-phenylserine; O—(O-acetylsalicyl)-threonine; N,O-di(O-acetylsalicyl)-threonine, O—(O-acetylsalicyl)-tyrosine; N,O-di(O-acetylsalicyl)-tyrosine, O—(O-acetylsalicyl)-dopa; O,O-di(O-acetylsalicyl)-dopa; N,O-di(O-acetylsalicyl)-dopa and N,O,O-tri(O-acetylsalicyl)-dopa; and mixtures and combinations thereof.

O-acetylsalicyl derivatives of aminocarbohydrates, amino acids and peptides of the embodiments have the advantages of similar pharmacological and therapeutic effects of aspirin, but without the adverse effects such as gastrointestinal bleeding. The pharmacological and therapeutic effects include analgesic, anti-inflammatory and antipyretic actions, and protection for cardiovascular system.

In accordance with preferred embodiments, a composition comprising at least one compound selected from the group consisting of the O-acetylsalicyl derivatives of aminocarbohydrates, amino acids and peptides, their free acids, esters, amides, lactones and salt forms present in a therapeutically effective amount and in a pharmaceutically acceptable vehicle for topical as well as systemic treatment of disorders associated with nervous, vascular, musculoskeletal or cutaneous system is provided.

In one embodiment, the composition may comprise a mixture of O-acetylsalicylic acid (aspirin) and an O-acetylsalicyl derivative, such as an O-acetylsalicyl aminocarbohydrate derivative comprising glucosamine or a basic amino acid such as arginine, ornithine or lysine. The composition may also comprise a mixture of O-acetylsalicyl derivative compounds, with or without the presence of aspirin. In a preferred embodiment, a composition may comprise one O-acetylsalicyl derivative compound, without the presence of aspirin or other O-acetylsalicyl derivative compound.

In another embodiment, the composition further comprises a cosmetic, pharmaceutical or other agent that can be administered at the same time or in sequence. These agents include hydroxyacids, ketoacids and related compounds; phenyl alpha acyloxyalkanoic acids and derivatives; N-acetyl-aldosamines, N-acetylamino acids and related N-acetyl compounds; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotion sickness agents; antiinflammatory agents; antihyperkeratotic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; astringents; cleansing agents; corn, callus and wart removing agents; topical cardiovascular agents; vitamins; corticosteroids; tanning agents; hormones; retinoids; gum disease or oral care agents.

The cosmetic, pharmaceutical and other agents that can be incorporated into the compositions comprising O-acetylsalicyl derivatives for synergetic or synergistic effects include: abacavir, acebutolol, acetaminophen, acetaminosalol, acetazolamide, acetohydroxamic acid, N-acetyl-cysteine, N-acetyl-glucosamine, N-acetyl-glutamine, N-acetyl-lysine, N-acetyl-ornithine, N-acetyl-prolinamide, N-acetyl-proline, acetylsalicylic acid, acitretin, aclovate, acrivastine, actiq, acyclovir, adapalene, adefovir dipivoxil, adenosine, albuterol, alfuzosin, allopurinol, alloxanthine, almotriptan, alprazolam, alprenolol, aluminum acetate, aluminum chloride, aluminum chlorohydroxide, aluminum hydroxide, amantadine, amiloride, aminacrine, aminobenzoic acid (PABA), aminocaproic acid, aminosalicylic acid, amiodarone, amitriptyline, amlodipine, amocarzine, amodiaquin, amorolfine, amoxapine, amphetamine, ampicillin, anagrelide, anastrozole, anthralin, apomorphine, aprepitant, arbutin, aripiprazole, ascorbic acid, ascorbyl palmitate, atazanavir, atenolol, atomoxetine, atropine, azathioprine, azelaic acid, azelastine, azithromycin, bacitracin, beclomethasone dipropionate, bemegride, benazepril, bendroflumethiazide, benzocaine, benzonatate, benzophenone, benzoyl peroxide, benztropine, bepridil, betamethasone dipropionate, betamethasone valerate, brimonidine, brompheniramine, bupivacaine, buprenorphine, bupropion, burimamide, butenafine, butoconazole, cabergoline, caffeic acid, caffeine, calcipotriene, camphor, candesartan cilexetil, capsaicin, carbamazepine, carbamide peroxide, cefditoren pivoxil, cefepime, cefpodoxime proxetil, celecoxib, cetirizine, cevimeline, chitosan, chlordiazepoxide, chlorhexidine, chloroquine, chlorothiazide, chloroxylenol, chlorpheniramine, chlorpromazine, chlorpropamide, ciclopirox, cilostazol, cimetidine, cinacalcet, ciprofloxacin, citalopram, citric acid, cladribine, clarithromycin, clemastine, clindamycin, clioquinol, clobetasol propionate, clomiphene, clonidine, clopidogrel, clotrimazole, clozapine, coal tar, cocaine, codeine, cromolyn, crotamiton, cyclizine, cyclobenzaprine, cycloserine, cytarabine, dacarbazine, dalfopristin, dapsone, daptomycin, daunorubicin, deferoxamine, dehydroepiandrosterone, delavirdine, desipramine, desloratadine, desmopressin, desoximetasone, dexamethasone, dexmedetomidine, dexmethylphenidate, dexrazoxane, dextroamphetamine, diazepam, dicyclomine, didanosine, dihydrocodeine, dihydromorphine, diltiazem, 6,8-dimercaptooctanoic acid (dihydrolipoic acid), diphenhydramine, diphenoxylate, dipyridamole, disopyramide, dobutamine, dofetilide, dolasetron, donepezil, dopa esters, dopamide, dopamine, dorzolamide, doxepin, doxorubicin, doxycycline, doxylamine, doxypin, duloxetine, dyclonine, econazole, eflornithine, eletriptan, emtricitabine, enalapril, ephedrine, epinephrine, epinine, epirubicin, eptifibatide, ergotamine, erythromycin, escitalopram, esmolol, esomeprazole, estazolam, estradiol, ethacrynic acid, ethinyl estradiol, etidocaine, etomidate, famciclovir, famotidine, felodipine, fentanyl, ferulic acid, fexofenadine, flecainide, fluconazole, flucytosine, fluocinolone acetonide, fluocinonide, 5-fluorouracil, fluoxetine, fluphenazine, flurazepam, fluvoxamine, formoterol, furosemide, galactarolactone, galactonic acid, galactonolactone, galantamine, gatifloxacin, gefitinib, gemcitabine, gemifloxacin, glucarolactone, gluconic acid, gluconolactone, glucuronic acid, glucuronolactone, glycolic acid, griseofulvin, guaifenesin, guanethidine, N-guanylhistamine, haloperidol, haloprogin, hexylresorcinol, homatropine, homosalate, hydralazine, hydrochlorothiazide, hydrocortisone, hydrocortisone 21-acetate, hydrocortisone 17-butyrate, hydrocortisone 17-valerate, hydrogen peroxide, hydromorphone, hydroquinone, hydroquinone monoether, hydroxyzine, hyoscyamine, hypoxanthine, ibuprofen, ichthammol, idarubicin, imatinib, imipramine, imiquimod, indinavir, indomethacin, irbesartan, irinotecan, isoetharine, isoproterenol, itraconazole, kanamycin, ketamine, ketanserin, ketoconazole, ketoprofen, ketotifen, kojic acid, labetalol, lactic acid, lactobionic acid, lamivudine, lamotrigine, lansoprazole, letrozole, leuprolide, levalbuterol, levofloxacin, lidocaine, linezolid, lobeline, loperamide, losartan, loxapine, lysergic diethylamide, mafenide, malic acid, maltobionic acid, mandelic acid, maprotiline, mebendazole, mecamylamine, meclizine, meclocycline, memantine, menthol, meperidine, mepivacaine, mercaptopurine, mescaline, metanephrine, metaproterenol, metaraminol, metformin, methadone, methamphetamine, methotrexate, methoxamine, methyldopa esters, methyldopamide, 3,4-methylenedioxymethamphetamine, methyllactic acid, methyl nicotinate, methylphenidate, methyl salicylate, metiamide, metolazone, metoprolol, metronidazole, mexiletine, miconazole, midazolam, midodrine, miglustat, minocycline, minoxidil, mirtazapine, mitoxantrone, moexiprilat, molindone, monobenzone, morphine, moxifloxacin, moxonidine, mupirocin, nadolol, naftifine, nalbuphine, nalmefene, naloxone, naproxen, nefazodone, nelfinavir, neomycin, nevirapine, nicardipine, nicotine, nifedipine, nimodipine, nisoldipine, nizatidine, norepinephrine, nystatin, octopamine, octreotide, octyl methoxycinnamate, octyl salicylate, ofloxacin, olanzapine, olmesartan medoxomil, olopatadine, omeprazole, ondansetron, oxiconazole, oxotremorine, oxybenzone, oxybutynin, oxycodone, oxymetazoline, padimate O, palonosetron, pantothenic acid, pantoyl lactone, paroxetine, pemoline, penciclovir, penicillamine, penicillins, pentazocine, pentobarbital, pentostatin, pentoxifylline, pergolide, perindopril, permethrin, phencyclidine, pheneizine, pheniramine, phenmetrazine, phenobarbital, phenol, phenoxybenzamine, phentolamine, phenylephrine, phenylpropanolamine, phenytoin, physostigmine, pilocarpine, pimozide, pindolol, pioglitazone, pipamazine, piperonyl butoxide, pirenzepine, podofilox, podophyllin, povidone iodine, pramipexole, pramoxine, prazosin, prednisone, prenalterol, prilocaine, procainamide, procaine, procarbazine, promazine, promethazine, promethazine propionate, propafenone, propoxyphene, propranolol, propylthiouracil, protriptyline, pseudoephedrine, pyrethrin, pyrilamine, pyrimethamine, quetiapine, quinapril, quinethazone, quinidine, quinupristin, rabeprazole, reserpine, resorcinol, retinal, 13-cis retinoic acid, retinoic acid, retinol, retinyl acetate, retinyl palmitate, ribavirin, ribonic acid, ribonolactone, rifampin, rifapentine, rifaximin, riluzole, rimantadine, risedronic acid, risperidone, ritodrine, rivastigmine, rizatriptan, ropinirole, ropivacaine, salicylamide, salicylic acid, salmeterol, scopolamine, selegiline, selenium sulfide, serotonin, sertindole, sertraline, shale tar, sibutramine, sildenafil, sotalol, streptomycin, strychnine, sulconazole, sulfabenz, sulfabenzamide, sulfabromomethazine, sulfacetamide, sulfachlorpyridazine, sulfacytine, sulfadiazine, sulfadimethoxine, sulfadoxine, sulfaguanole, sulfalene, sulfamethizole, sulfamethoxazole, sulfanilamide, sulfapyrazine, sulfapyridine, sulfasalazine, sulfasomizole, sulfathiazole, sulfisoxazole, sulfur, tadalafil, tamsulosin, tartaric acid, tazarotene, tegaserol, telithromycin, telmisartan, temozolomide, tenofovir disoproxil, terazosin, terbinafine, terbutaline, terconazole, terfenadine, tetracaine, tetracycline, tetrahydrozoline, theobromine, theophylline, thiabendazole, thioctic acid (lipoic acid), thioridazine, thiothixene, thymol, tiagabine, timolol, tinidazole, tioconazole, tirofiban, tizanidine, tobramycin, tocainide, tolazoline, tolbutamide, tolnaftate, tolterodine, tramadol, tranylcypromine, trazodone, triamcinolone acetonide, triamcinolone diacetate, triamcinolone hexacetonide, triamterene, triazolam, triclosan, triflupromazine, trimethoprim, trimipramine, tripelennamine, triprolidine, tromethamine, tropic acid, tyramine, undecylenic acid, urea, urocanic acid, ursodiol, vardenafil, venlafaxine, verapamil, vitamin E acetate, voriconazole, warfarin, wood tar, xanthine, zafirlukast, zaleplon, zinc pyrithione, ziprasidone, zolmitriptan, zolpidem

Compositions comprising O-acetylsalicyl derivatives can be formulated as solution, gel, lotion, cream, ointment, shampoo, spray, stick, powder, masque, mouth rinse or wash, vaginal gel or preparation, or other form acceptable for use on skin, nail, hair, oral mucosa, vaginal or anal mucosa, mouth or gums.

To prepare a solution composition, at least one O-acetylsalicyl derivative of the embodiments can be dissolved in a solution prepared from water, ethanol, propylene glycol, butylene glycol, and/or other topically acceptable vehicles. The concentration of a single O-acetylsalicyl derivative or the total concentration of all O-acetylsalicyl derivatives where the composition comprises more than one O-acetylsalicyl derivative, can range from 0.01 to 99.9% by weight of the total composition, with preferred concentrations of from about 0.1 to about 50% by weight of the total composition and with more preferred concentration of from about 0.5 to about 25% by weight of the total composition.

To prepare a topical composition in lotion, cream or ointment form, the O-acetylsalicyl derivative can first be dissolved in water, ethanol, propylene glycol, and/or other vehicles, and the solution thus obtained mixed with a desired base or pharmaceutically acceptable vehicle to make a lotion, cream or ointment. Concentrations of the O-acetylsalicyl derivative can be the same as described above.

A topical composition also can be formulated in a gel or shampoo form. A typical gel composition can be formulated by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate to a solution comprising the O-acetylsalicyl derivative. The preferred concentration of the gelling agent may range from about 0.1 to about 4 percent by weight of the total composition. In the preparation of shampoo, the O-acetylsalicyl derivative can first be dissolved in water or propylene glycol, and the solution thus obtained is mixed with a shampoo base. Concentrations of the O-acetylsalicyl derivative used in gel or shampoo form can be the same as described above.

To prepare a topical combination composition for synergetic or synergistic effects, a cosmetic, pharmaceutical or other agent can be incorporated into any one of the above compositions by dissolving or mixing the agent into the formulation. Other forms of compositions for delivery of O-acetylsalicyl derivatives are readily blended, prepared or formulated by those skilled in the art.

For systemic use the O-acetylsalicyl derivative can be formulated for oral administration or for parenteral injections. In oral preparations, the O-acetylsalicyl derivative can be formulated in tablet form or in gelatin capsules with or without mixing with gelatin powder. Each tablet or capsule can contain from about 10 to about 300 mg of the O-acetylsalicyl derivative. For example, Lilly gelatin capsules, sizes 1 and 0, contained approximately 100 mg and 150 mg, respectively, of N—(O-acetylsalicyl)-L-tyrosine ethyl ester powder. In the same manner, Lilly gelatin capsules, sizes 1 and 0, contained approximately 130 mg and 200 mg, respectively, of N—(O-acetylsalicyl)-D-4-hydroxyphenylglycine ethyl ester powder. For parenteral injections the O-acetylsalicyl derivative can be prepared under sterilized conditions, usually in about 1 to about 10% concentration in water, propylene glycol and/or other pharmaceutically acceptable vehicle.

A composition comprising an O-acetylsalicyl derivative of the embodiments can be administered topically or systemically to a human subject in need for prevention or treatment of cosmetic conditions, dermatological disorders, or diseases associated with nervous, vascular, musculoskeletal or cutaneous system.

For topical administration, a composition comprising the O-acetylsalicyl derivative as prepared according to the preparation section can be topically applied one to three times, preferably twice daily to the lesions or the cutaneous sites associated with disorders or diseases. The topical application can continue until the disorder or disease has been eradicated or substantially improved. The treatment period depends on the condition or severity of the disorder or disease, and also depends on the individual subject. Examples of disorders and diseases associated with the nervous, vascular, musculoskeletal or cutaneous systems include pain, pruritus, inflammation, erythema, dermatitis, acne, eczema, severe dry skin, ichthyosis, calluses, keratoses, hyperkeratotic conditions, age spots, psoriasis, wrinkles and photoaging skin. Compositions comprising the O-acetylsalicyl derivative of the present invention have been found to be beneficial or effective for topical prevention or treatment of various disorders associated with the nervous, vascular, musculoskeletal or cutaneous systems. For example, a human subject with severe dry skin (ichthyosis) topically applied N—(O-acetylsalicyl)-glucosamine (5%) in hydrophilic ointment twice daily for two weeks. At the end of two week treatment, the thick and scaly skin became smooth and the skin exhibited a 75% improvement, as judged by medical evaluation.

For systemic administration, a composition comprising the O-acetylsalicyl derivative as prepared according to the preparation section can be administered by injection, infusion or oral intake, with a preferred route of oral administration. A composition comprising the O-acetylsalicyl derivative can be taken orally one to three times, preferably twice daily for prevention or treatment of disorders and diseases associated with the nervous, vascular, musculoskeletal or cutaneous systems. The oral administration can continue until the disorder or disease has been eradicated or substantially improved.

We have found that a composition comprising an O-acetylsalicyl derivative administered systemically is therapeutically beneficial or effective for prevention or treatment of disorders and diseases associated with the nervous, vascular, musculoskeletal or cutaneous systems. The disorders and diseases include pain, pruritus, inflammation, erythema, dermatitis, eczema, dementia, joint pain or swelling and arthritis. For example, a human subject having osteoarthritis of both knees for 4 years had symptoms of pain upon walking and at rest ranging in pain intensity from moderate to severe. The symptoms had not been relieved by nonsteroidal anti-inflammatory agents such as naproxen and ibuprofen, but had been relieved incompletely by oral prednisone, 10-30 mg daily. The subject had taken oral aspirin a few years ago, but had encountered acute gastrointestinal bleeding after oral aspirin. The subject took an oral dose of 100 mg N—(O-acetylsalicyl)-L-tyrosine ethyl ester. There was negligible discomfort or adverse reactions with this dosage and the relief of knee pain was limited. When the oral dose was increased to 1 g, the knee pain disappeared after two and half hours of oral administration. The subject did intensive physical activities without feeling knee pain for the entire day. There was negligible discomfort or adverse reactions caused by the 1 g oral dose of N—(O-acetylsalicyl)-L-tyrosine ethyl ester. The above result reveal that oral O-acetylsalicyl amino acids would be therapeutically effective for treatment of disorders and diseases associated with nervous, vascular, musculoskeletal or cutaneous system.

Among the compounds described herein, the preferred O-acetylsalicyl derivatives include: N—(O-acetylsalicyl)-glucosamine, N—(O-acetylsalicyl)-mannosamine, N—(O-acetylsalicyl)-galactosamine, N—(O-acetylsalicyl)-glucamine, N—(O-acetylsalicyl)-glycine ethyl ester, N—(O-acetylsalicyl)-tyrosine ethyl ester, N—(O-acetylsalicyl)-proline, N—(O-acetylsalicyl)-prolinamide, N—(O-acetylsalicyl)-proline ethyl ester, N—(O-acetylsalicyl)-glutamine, N—(O-acetylsalicyl)-glutamic acid, N—(O-acetylsalicyl)-glutamic acid diethyl ester, N—(O-acetylsalicyl)-arginine, N—(O-acetylsalicyl)-4-hydroxyphenylglycine ethyl ester; N,O-di(O-acetylsalicyl)-4-hydroxyphenylglycine ethyl ester, N—(O-acetylsalicyl)-3-phenylserine; N—(O-acetylsalicyl)-3-phenylserine ethyl ester; N—(O-acetylsalicyl)-lysine, N—(O-acetylsalicyl)-ornithine, and mixtures and combinations thereof. These representative compounds would be effective and beneficial for oral administration to relieve pain and inflammation of the joints caused by arthritis or other disorders.

The following examples are illustrative, but not limiting, of the methods and compositions of the present invention. Other suitable modifications and adaptations of the variety of conditions and parameters normally encountered in therapy and that are obvious to those skilled in the art are within the spirit and scope of the embodiments.

### EXAMPLE 1

N—(O-Acetylsalicyl)-D-glucosamine 5 g was dissolved in warm propylene glycol 35 ml, and the solution thus obtained was mixed with hydrophilic ointment or oil-in-water cream (60 g). The cream or lotion composition thus prepared had pH 4.6 and contained 5% N—(O-acetylsalicyl)-D-glucosamine.

A male subject, age 73, having itchy eczema on his right leg, topically applied the above 5% N—(O-acetylsalicyl)-D-glucosamine cream. The itch subsided about 5 minutes after the topical application. Therapeutic evaluation was judged to be 75% effective for immediate relief of itch. The results showed that N—(O-acetylsalicyl)-aminocarbohydrates would be therapeutically beneficial or effective for topical treatment of disorders associated with nervous, vascular, musculoskeletal or cutaneous system.

### EXAMPLE 2

N—(O-Acetylsalicyl)-glycine ethyl ester 5 g was dissolved in 95 ml solution prepared from ethanol 70 parts and propylene glycol 30 parts by volume. The solution composition thus prepared, pH 5.3, contained 5% N—(O-acetylsalicyl)-glycine ethyl ester, and should be therapeutically beneficial or effective for topical treatment of inflammation, and disorders associated with nervous, vascular, musculoskeletal or cutaneous system.

### EXAMPLE 3

N—(O-Acetylsalicyl)-glycine ethyl ester 5 g was dissolved in warm propylene glycol 35 ml, and the solution thus obtained was mixed with hydrophilic ointment or oil-in-water cream (60 g). The cream or lotion composition thus prepared had pH 4.4 and contained 5% N—(O-acetylsalicyl)-glycine ethyl ester.

A male subject, age 73, having eczema on his left leg, topically applied the above 5% N—(O-acetylsalicyl)-glycine ethyl ester cream. The itch subsided about 5 minutes after the topical application. Therapeutic evaluation was judged to be 75% effective for immediate relief of itch. The results showed that N—(O-acetylsalicyl)-amino acid esters would be therapeutically beneficial or effective for topical treatment of disorders associated with nervous, vascular, musculoskeletal or cutaneous system.

### EXAMPLE 4

N—(O-Acetylsalicyl)-L-tyrosine ethyl ester 5 g was dissolved in 95 ml solution prepared from ethanol 70 parts and propylene glycol 30 parts by volume. The solution composition thus prepared had pH 3.6 and contained 5% N—(O-acetylsalicyl)-L-tyrosine ethyl ester, and should be therapeutically beneficial or effective for topical treatment of pain, inflammation, and diseases associated with nervous, vascular, musculoskeletal or cutaneous system.

### EXAMPLE 5

N—(O-Acetylsalicyl)-L-tyrosine ethyl ester 5 g was dissolved in warm propylene glycol 35 ml, and the solution thus obtained was mixed with hydrophilic ointment or oil-in-water cream (60 g). The cream or lotion composition thus prepared had pH 3.0 and contained 5% N—(O-acetylsalicyl)-L-tyrosine ethyl ester.

A male subject, age 73, having itchy eczema on his left thigh, topically applied the above 5% N—(O-acetylsalicyl)-L-tyrosine ethyl ester cream. The itch subsided about 5 minutes after the topical application. Therapeutic evaluation was judged to be 75% effective for immediate relief of itch. The results showed that N—(O-acetylsalicyl)-amino acid esters would be therapeutically beneficial or effective for topical treatment of disorders associated with nervous, vascular, musculoskeletal or cutaneous system.

### EXAMPLE 6

N—(O-Acetylsalicyl)-L-proline 5 g was dissolved in 95 ml solution prepared from ethanol 70 parts and propylene glycol 30 parts by volume. The solution composition thus prepared with pH 2.2, contained 5% N—(O-acetylsalicyl)-L-proline, and should be therapeutically beneficial or effective for topical treatment of itch, inflammation, and disorders associated with nervous, vascular, musculoskeletal or cutaneous system.

### EXAMPLE 7

N—(O-Acetylsalicyl)-L-proline 5 g was dissolved in warm propylene glycol 35 ml, and the solution thus obtained was mixed with hydrophilic ointment or oil-in-water cream (60 g). The cream or lotion composition thus prepared had pH 2.5 and contained 5% N—(O-acetylsalicyl)-L-proline.

A male subject, age 73, having itchy eczema on the back of his left hand, topically applied the above 5% N—(O-acetylsalicyl)-L-proline cream. The itch subsided about 5 minutes after the topical application. Therapeutic evaluation was judged to be 100% effective for immediate relief of itch. The results showed that N—(O-acetylsalicyl)-amino acids would be therapeutically beneficial or effective for topical treatment of disorders associated with nervous, vascular, musculoskeletal or cutaneous system.

### EXAMPLE 8

N—(O-Acetylsalicyl)-D-4-hydroxyphenylglycine ethyl ester 5 g was dissolved in warm propylene glycol 35 ml, and the soft paste thus obtained was mixed with hydrophilic ointment or oil-in-water cream (60 g). The cream or lotion composition thus prepared with pH 3.1, contained 5% N—(O-acetylsalicyl)-D-4-hydroxyphenylglycine ethyl ester, and should be therapeutically beneficial or effective for topical treatment of severe dry skin, inflammation, and disorders associated with nervous, vascular, musculoskeletal or cutaneous system.

### EXAMPLE 9

A male subject, age 34, having severe dry skin condition of X-linked ichthyosis with thick and adherent scales topically applied twice daily 5% N—(O-acetylsalicyl)-D-glucosamine cream as prepared in Example 1 for 2 weeks. At the end of two weeks, the adherent scales in the treated areas were reduced and the treated skin became smooth. The improvement was judged to be 75% by clinical evaluation.

### EXAMPLE 10

Male subject, age 83, weight 90 kg, had 4 year history of osteoarthritis of both knees which was confirmed by X-ray and MRI showing diminished articular space. Symptoms of pain upon walking and at rest, as well as tenderness to palpation, had been consistently present, varying in intensity from moderate to severe. The symptoms had not been relieved by nonsteroidal anti-inflammatory agents such as naproxen and ibuprofen, but had been relieved incompletely by oral prednisone, 10-30 mg daily.

The subject administered an oral dose of N—(O-acetylsalicyl)-L-tyrosine ethyl ester, 10 mg in the morning and 100 mg in the afternoon. There was negligible discomfort or adverse reactions with this dosage and the relief of knee pain was limited. When the oral dose was increased to 1 g, the knee pain subsided after two and half hours of oral administration. The subject did intensive physical activities during the next 10 hours that consisted of farming manual labor that included climbing up and down the banks of a pond to cut off cattail weeds at root level, and manually carrying stalks up the pond bank to level ground. Other physical exertions included numerous mounting and demounting from a farm tractor that pulled a trailer containing cattail weeds to dumping sites. During the entire day and into the evening, the presence of knee pain or impaired knee function was negligible. Discomfort did re-appear by the next morning. The subject administered an oral dose of 0.5 g, and over the ensuing 12 hours there was incomplete relief of knee discomfort. When the oral dose was increased to 0.7 g, there was marked, but incomplete, relief of knee discomfort.

The daily oral dose of the O-acetylsalicyl derivatives described herein, most preferably N—(O-acetylsalicyl)-L-tyrosine ethyl ester, can range from about 5 to about 30 mg/kg, preferably about 7 to about 20 mg/kg, and most preferably from about 10 to about 15 mg/kg body weight.

The above results show that N—(O-acetylsalicyl)-amino acids should be therapeutically beneficial or effective for systemic treatment of pain, inflammation, and diseases associated with nervous, vascular, musculoskeletal or cutaneous system.

### EXAMPLE 11

N—(O-Acetylsalicyl)-glutamic acid diethyl ester 5 g was dissolved in warm propylene glycol 35 ml, and the solution thus obtained was mixed with hydrophilic ointment or oil-in-water cream 60 g. The cream or lotion composition thus prepared had pH 5.2 and contained 5% N—(O-acetylsalicyl)-glutamic acid diethyl ester.

A male subject, age 73, having eczema on his right leg with intensive itch, topically applied the above 5% N—(O-acetylsalicyl)-glutamic acid diester cream to skin lesions. The itch subsided about 5 minutes after the topical application. Therapeutic evaluation was judged to be 100% effective for immediate relief of itch. The results showed that N—(O-acetylsalicyl)-amino acid esters would be therapeutically beneficial or effective for topical treatment of disorders associated with nervous, vascular, musculoskeletal or cutaneous system.

### EXAMPLE 12

N—(O-Acetylsalicyl)-D-galactosamine 5 g was dissolved in warm propylene glycol 35 ml, and the solution thus obtained was mixed with hydrophilic ointment or oil-in-water cream (60 g). The cream or lotion composition thus prepared had pH 3.9 and contained 5% N—(O-acetylsalicyl)-D-galactosamine.

A male subject, age 73, having itchy eczema on his right leg, topically applied the above 5% N—(O-acetylsalicyl)-D-galactosamine cream. The itch subsided about 5 minutes after the topical application. Therapeutic evaluation was judged to be 90% effective for immediate relief of itch. The results showed that N—(O-acetylsalicyl)-aminocarbohydrates would be therapeutically beneficial or effective for topical treatment of disorders associated with nervous, vascular, musculoskeletal or cutaneous system.

### EXAMPLE 13

N—(O-Acetylsalicyl)-L-proline methyl ester 5 g was dissolved in warm propylene glycol 35 ml, and the solution thus obtained was mixed with hydrophilic ointment or oil-in-water cream (60 g). The cream or lotion composition thus prepared had pH 4.9, and contained 5% N—(O-acetylsalicyl)-L-proline methyl ester.

A male subject, age 73, having itchy eczema on the left leg, topically applied the above 5% N—(O-acetylsalicyl)-L-proline methyl ester cream. The itch subsided in about 2 to 3 minutes after the topical application. Therapeutic evaluation was judged to be 90% effective for immediate relief of itch. The results showed that N—(O-acetylsalicyl)-amino acid esters would be therapeutically beneficial or effective for topical treatment of disorders associated with nervous, vascular, musculoskeletal or cutaneous system.

While the invention has been described with reference to particularly preferred embodiments and examples, those skilled in the art recognize that various modifications may be made to the invention without departing from the spirit and scope thereof.

1. A composition comprising a therapeutically effective amount of at least one O-acetylsalicyl derivative and a pharmaceutically acceptable excipient or vehicle, wherein the O-acetylsalicyl derivative is selected from the group consisting of O-acetylsalicyl aminocarbohydrates, O-acetylsalicyl amino acids, O-acetylsalicyl peptides, as free acid, salt, partial salt, lactone, amide, ester, stereoisomeric and non-stereoisomeric forms thereof.

2. The composition of claim 1, wherein the O-acetylsalicyl derivative is selected from at least one of the group consisting of N—(O-acetylsalicyl)-glucosamine, N—(O-acetylsalicyl)-mannosamine, N—(O-acetylsalicyl)-galactosamine, N—(O-acetylsalicyl)-glucamine N—(O-acetylsalicyl)-glycine ethyl ester, N—(O-acetylsalicyl)-tyrosine ethyl ester, N—(O-acetylsalicyl)-proline, N—(O-acetylsalicyl)-prolinamide, N—(O-acetylsalicyl)-proline ethyl ester, N—(O-acetylsalicyl)-glutamine, N—(O-acetylsalicyl)-glutamic acid, N—(O-acetylsalicyl)-glutamic acid diethyl ester, N—(O-acetylsalicyl)-arginine, N—(O-acetylsalicyl)-4-hydroxyphenylglycine ethyl ester; N,O-di(O-acetylsalicyl)-4-hydroxyphenylglycine ethyl ester, N—(O-acetylsalicyl)-3-phenylserine; N—(O-acetylsalicyl)-3-phenylserine ethyl ester; N—(O-acetylsalicyl)-lysine and N—(O-acetylsalicyl)-ornithine as free acid, salt, partial salt, lactone, amide, ester, stereoisomeric and non-stereoisomeric forms thereof.

3. The composition of claim 1, wherein the O-acetylsalicyl aminocarbohydrate has the formula:
R₁(CHOH)ₙ(CHNHR₂)R₃,
where n is an integer, preferably 1-9; R₁ is selected from the group consisting of H, I, F, Cl, Br, CHO, CONH₂, COOR₄, an alkyl, alkoxyl, aralkyl or aryl group having 1 to 19 carbon atoms; R₂ is a radical group of O-acetylsalicyl, represented by CH₃COOC₆H₄CO; R₃ is selected from the group consisting of H, CHO, CH₂OH, CONH₂, and COOR₄; and R₄ is independently selected from the group consisting of H, an alkyl, aralkyl or aryl group having 1 to 9 carbon atoms; the H attached to any carbon atom can be substituted by I, F, Cl, Br, SH, CHO, CONH₂, NH₂, alkyl, alkoxyl, aralkyl or aryl group having 1 to 9 carbon atoms; and the OH can be substituted by H, NH₂, NHCOCH₃, acetoxyl or other acyloxyl group having 2 to 9 carbon atoms.

4. The composition of claim 3, wherein the O-acetylsalicyl aminocarbohydrate is selected from at least one of the group consisting of N—(O-acetylsalicyl)-glycerosamine, N—(O-acetylsalicyl)-erythrosamine, N—(O-acetylsalicyl)-threosamine, N—(O-acetylsalicyl)-ribosamine, N—(O-acetylsalicyl)-arabinosamine, N—(O-acetylsalicyl)-xylosamine, N—(O-acetylsalicyl)-lyxosamine, N—(O-acetylsalicyl)-allosamine, N—(O-acetylsalicyl)-altrosamine, N—(O-acetylsalicyl)-glucosamine, N—(O-acetylsalicyl)-mannosamine, N—(O-acetylsalicyl)-gulosamine, N—(O-acetylsalicyl)-idosamine, N—(O-acetylsalicyl)-galactosamine, N—(O-acetylsalicyl)-talosamine, N—(O-acetylsalicyl)-alloheptosamine, N—(O-acetylsalicyl)-altroheptosamine, N—(O-acetylsalicyl)-glucoheptosamine, N—(O-acetylsalicyl)-mannoheptosamine, N—(O-acetylsalicyl)-guloheptosamine, N—(O-acetylsalicyl)-idoheptosamine, N—(O-acetylsalicyl)-galactoheptosamine, N—(O-acetylsalicyl)-taloheptosamine, N—(O-acetylsalicyl)-glyceraminic acid, N—(O-acetylsalicyl)-erythrosaminic acid, N—(O-acetylsalicyl)-threosaminic acid, N—(O-acetylsalicyl)-ribosaminic acid, N—(O-acetylsalicyl)-arabinosaminic acid, N—(O-acetylsalicyl)-xylosaminic acid, N—(O-acetylsalicyl)-lyxosaminic acid, N—(O-acetylsalicyl)-allosaminic acid, N—(O-acetylsalicyl)-altrosaminic acid, N—(O-acetylsalicyl)-glucosaminic acid, N—(O-acetylsalicyl)-mannosaminic acid, N—(O-acetylsalicyl)-gulosaminic acid, N—(O-acetylsalicyl)-idosaminic acid, N—(O-acetylsalicyl)-galactosaminic acid, N—(O-acetylsalicyl)-talosaminic acid, N—(O-acetylsalicyl)-alloheptosaminic acid, N—(O-acetylsalicyl)-altroheptosaminic acid, N—(O-acetylsalicyl)-glucoheptosaminic acid, N-(O-acetylsalicyl)-mannoheptosaminic acid, N—(O-acetylsalicyl)-guloheptosaminic acid, N—(O-acetylsalicyl)-idoheptosaminic acid, N—(O-acetylsalicyl)-galactoheptosaminic acid, N—(O-acetylsalicyl)-taloheptosaminic acid, N—(O-acetylsalicyl)lactosamine, N—(O-acetylsalicyl)muramic acid, N—(O-acetylsalicyl)neuramine, N—(O-acetylsalicyl)neuramin lactose, N—(O-acetylsalicyl)-neuraminic acid. N—(O-acetylsalicyl)-4-O-acetylneuraminic acid, N—(O-acetylsalicyl)-7-O-acetylneuraminic acid, N—(O-acetylsalicyl)-di-O-acetylneuraminic acid, N—(O-acetylsalicyl)-glycerosylamine, N—(O-acetylsalicyl)-erythrosylamine, N—(O-acetylsalicyl)-threosylamine, N—(O-acetylsalicyl)-ribosylamine, N—(O-acetylsalicyl)-arabinosylamine, N—(O-acetylsalicyl)-xylosylamine, N—(O-acetylsalicyl)-lyxosylamine, N—(O-acetylsalicyl)-allosylamine, N—(O-acetylsalicyl)-altrosylamine, N—(O-acetylsalicyl)-glucosylamine [same as N—(O-acetylsalicyl)-glucamine], N—(O-acetylsalicyl)-mannosylamine, N—(O-acetylsalicyl)-gulosylamine, N—(O-acetylsalicyl)-idosylamine, N—(O-acetylsalicyl)-galactosylamine, N—(O-acetylsalicyl)-talosylamine, N—(O-acetylsalicyl)-alloheptosylamine, N—(O-acetylsalicyl)-altroheptosylamine, N—(O-acetylsalicyl)-glucoheptosylamine, N—(O-acetylsalicyl)-mannoheptosylamine, N—(O-acetylsalicyl)-guloheptosylamine, N—(O-acetylsalicyl)-idoheptosylamine, N—(O-acetylsalicyl)-galactoheptosylamine, and N—(O-acetylsalicyl)-taloheptosylamine.

5. The composition of claim 1, wherein the O-acetylsalicyl aminocarbohydrate has the formula:
R₁(CHOR₂)ₙ(CHNH₂) R₃,
where n is an integer, preferably 1-9; R₁ is selected from the group consisting of H, I, F, Cl, Br, CHO, CONH₂, COOR₄, an alkyl, alkoxyl, aralkyl or aryl group having 1 to 19 carbon atoms; R₂ is H or at least one R₂ is a radical group of O-acetylsalicyl, represented by CH₃COOC₆H₄CO; R₃ is selected from the group consisting of H, CHO, CH₂OH, CONH₂, and COOR₄; and R₄ is independently selected from the group consisting of H, an alkyl, aralkyl or aryl group having 1 to 9 carbon atoms; the H attached to any carbon atom can be substituted by I, F, Cl, Br, SH, CHO, CONH₂, NH₂, alkyl, alkoxyl, aralkyl or aryl group having 1 to 9 carbon atoms; and the OH can be substituted by H, NH₂, NHCOCH₃, acetoxyl or other acyloxyl group having 2 to 9 carbon atoms.

6. The composition of claim 5, wherein the O-acetylsalicyl aminocarbohydrate is selected from at least one of the group consisting of O—(O-acetylsalicyl)-glycerosamine, O—(O-acetylsalicyl)-erythrosamine, O—(O-acetylsalicyl)-threosamine, O—(O-acetylsalicyl)-ribosamine, O—(O-acetylsalicyl)-arabinosamine, O—(O-acetylsalicyl)-xylosamine, O—(O-acetylsalicyl)-lyxosamine, O—(O-acetylsalicyl)-allosamine, O—(O-acetylsalicyl)-altrosamine, O—(O-acetylsalicyl)-glucosamine, O—(O-acetylsalicyl)-mannosamine, O—(O-acetylsalicyl)-gulosamine, O—(O-acetylsalicyl)-idosamine, O—(O-acetylsalicyl)-galactosamine, O—(O-acetylsalicyl)-talosamine, O—(O-acetylsalicyl)-alloheptosamine, O—(O-acetylsalicyl)-altroheptosamine, O—(O-acetylsalicyl)-glucoheptosamine, O—(O-acetylsalicyl)-mannoheptosamine, O—(O-acetylsalicyl)-guloheptosamine, O—(O-acetylsalicyl)-idoheptosamine, O—(O-acetylsalicyl)-galactoheptosamine, O—(O-acetylsalicyl)-taloheptosamine, O—(O-acetylsalicyl)-glyceraminic acid, O—(O-acetylsalicyl)-erytherosaminic acid, O—(O-acetylsalicyl)-threosaminic acid, O—(O-acetylsalicyl)-ribosaminic acid, O—(O-acetylsalicyl)-arabinosaminic acid, O—(O-acetylsalicyl)-xylosaminic acid, O—(O-acetylsalicyl)-lyxosaminic acid, O—(O-acetylsalicyl)-allosaminic acid, O—(O-acetylsalicyl)-altrosaminic acid, O—(O-acetylsalicyl)-glucosaminic acid, O—(O-acetylsalicyl)-mannosaminic acid, O—(O-acetylsalicyl)-gulosaminic acid, O—(O-acetylsalicyl)-idosaminic acid, O—(O-acetylsalicyl)-galactosaminic acid, O—(O-acetylsalicyl)-talosaminic acid, O—(O-acetylsalicyl)-alloheptosaminic acid, O—(O-acetylsalicyl)-altroheptosaminic acid, O—(O-acetylsalicyl)-glucoheptosaminic acid, O—(O-acetylsalicyl)-mannoheptosaminic acid, O—(O-acetylsalicyl)-guloheptosaminic acid, O—(O-acetylsalicyl)-idoheptosaminic acid, O—(O-acetylsalicyl)-galactoheptosaminic acid, O—(O-acetylsalicyl)-taloheptosaminic acid, O—(O-acetylsalicyl)lactosamine, O—(O-acetylsalicyl)muramic acid, O—(O-acetylsalicyl)neuramine, O—(O-acetylsalicyl)neuramin lactose, O—(O-acetylsalicyl)-neuraminic acid. O—(O-acetylsalicyl)-4-O-acetylneuraminic acid, O—(O-acetylsalicyl)-7-O-acetylneuraminic acid , O—(O-acetylsalicyl)-di-O-acetylneuraminic acid, O—(O-acetylsalicyl)-glycerosylamine, O—(O-acetylsalicyl)-erythrosylamine, O—(O-acetylsalicyl)-thgreosylamine, O—(O-acetylsalicyl)-ribosylamine, O—(O-acetylsalicyl)-arabinosylamine, O—(O-acetylsalicyl)-xylosylamine, O—(O-acetylsalicyl)-lyxosylamine, O—(O-acetylsalicyl)-allosylamine, O—(O-acetylsalicyl)-altrosylamme, acetylsalicyl)-glucosylamine [same as O—(O-acetylsalicyl)-glucamine], O—(O-acetylsalicyl)-mannosylamine, O—(O-acetylsalicyl)-gulosylamine, O—(O-acetylsalicyl)-idosylamine, O—(O-acetylsalicyl)-galactosylamine, O—(O-acetylsalicyl)-talosylamine, O—(O-acetylsalicyl)-alloheptosylamine, O—(O-acetylsalicyl)-altroheptosylamine, O—(O-acetylsalicyl)-glucoheptosylamine, O—(O-acetylsalicyl)-mannoheptosylamine, O—(O-acetylsalicyl)-guloheptosylamine, O—(O-acetylsalicyl)-idoheptosylamine, O—(O-acetylsalicyl)-galactoheptosylamine, and O—(O-acetylsalicyl)-taloheptosylamine.

7. The composition of claim 1, wherein the O-acetylsalicyl aminocarbohydrate has the formula:
R₁(CHOR₂)ₙ(CHNHCOC₆H₄OCOCH₃)R₃,
where n is an integer, preferably 1-9; R₁ is selected from the group consisting of H, I, F, Cl, Br, CHO, CONH₂, COOR₄, an alkyl, alkoxyl, aralkyl or aryl group having 1 to 19 carbon atoms; R₂ is H or at least one R₂ is a radical group of O-acetylsalicyl, represented by CH₃COOC₆H₄CO; R₃ is selected from the group consisting of H, CHO, CH₂OH, CONH₂, and COOR₄; and R₄ is independently selected from the group consisting of H, an alkyl, aralkyl or aryl group having 1 to 9 carbon atoms; and the H attached to any carbon atom can be substituted by I, F, Cl, Br, SH, CHO, CONH₂, NH₂, alkyl, alkoxyl, aralkyl or aryl group having 1 to 9 carbon atoms.

8. The composition of claim 7, wherein the said O-acetylsalicyl aminocarbohydrate is selected from at least one of the group consisting of N,O-di(O-acetylsalicyl)-glycerosamine; N,O-di(O-acetylsalicyl)-erythrosamine; N,O-di(O-acetylsalicyl)-threosamine; N,O-di(O-acetylsalicyl)-ribosamine; N,O-di(O-acetylsalicyl)-arabinosamine; N,O-di(O-acetylsalicyl)-xylosamine; N,O-di(O-acetylsalicyl)-Iyxosamine; N,O-di(O-acetylsalicyl)-allosamine; N,O-di(O-acetylsalicyl)-altrosamine; N,O-di(O-acetylsalicyl)-glucosamine; N,O-di(O-acetylsalicyl)-mannosamine; N,O-di(O-acetylsalicyl)-gulosamine; N,O-di(O-acetylsalicyl)-idosamine; N,O-di(O-acetylsalicyl)-galactosamine; N,O-di(O-acetylsalicyl)-talosamine; N,O-di(O-acetylsalicyl)-alloheptosamine; N,O-di(O-acetylsalicyl)-altroheptosamine; N,O-di(O-acetylsalicyl)-glucoheptosamine; N,O-di(O-acetylsalicyl)-mannoheptosamine; N,O-di(O-acetylsalicyl)-guloheptosamine; N,O-di(O-acetylsalicyl)-idoheptosamine; N,O-di(O-acetylsalicyl)-galactoheptosamine; N,O-di(O-acetylsalicyl)-taloheptosamine; N,O-di(O-acetylsalicyl)-glyceraminic acid; N,O-di(O-acetylsalicyl)-erythrosaminic acid; N,O-di(O-acetylsalicyl)-threosaminic acid; N,O-di(O-acetylsalicyl)-ribosaminic acid; N,O-di(O-acetylsalicyl)-arabinosaminic acid; N,O-di(O-acetylsalicyl)-xylosaminic acid; N,O-di(O-acetylsalicyl)-lyxosaminic acid; N,O-di(O-acetylsalicyl)-allosaminic acid; N,O-di(O-acetylsalicyl)-altrosaminic acid; N,O-di(O-acetylsalicyl)-glucosaminic acid; N,O-di(O-acetylsalicyl)-mannosaminic acid; N,O-di(O-acetylsalicyl)-gulosaminic acid; N,O-di(O-acetylsalicyl)-idosaminic acid; N,O-di(O-acetylsalicyl)-galactosaminic acid; N,O-di(O-acetylsalicyl)-talosaminic acid; N,O-di(O-acetylsalicyl)-alloheptosaminic acid; N,O-di(O-acetylsalicyl)-altroheptosaminic acid; N,O-di(O-acetylsalicyl)-glucoheptosaminic acid; N,O-di(O-acetylsalicyl)-mannoheptosaminic acid; N,O-di(O-acetylsalicyl)-guloheptosaminic acid; N,O-di(O-acetylsalicyl)-idoheptosaminic acid; N,O-di(O-acetylsalicyl)-galactoheptosaminic acid; N,O-di(O-acetylsalicyl)-taloheptosaminic acid; N,O-di(O-acetylsalicyl)lactosamine; N,O-di(O-acetylsalicyl)muramic acid; N,O-di(O-acetylsalicyl)neuramine; N,O-di(O-acetylsalicyl)neuramin lactose; N,O-di(O-acetylsalicyl)-neuraminic acid; N,O-di(O-acetylsalicyl)-4-O-acetylneuraminic acid; N,O-di(O-acetylsalicyl)-7-O-acetylneuraminic acid; N,O-di(O-acetylsalicyl)-di-O-acetylneuraminic acid, N,O-di(O-acetylsalicyl)-glycerosylamine; N,O-di(O-acetylsalicyl)-erythrosylamine; N,O-di(O-acetylsalicyl)-threosylamine; N,O-di(O-acetylsalicyl)-ribosylamine; N,O-di(O-acetylsalicyl)-arabinosylamine; N,O-di(O-acetylsalicyl)-xylosylamine; N,O-di(O-acetylsalicyl)-lyxosylamine; N,O-di(O-acetylsalicyl)-allosylamine; N,O-di(O-acetylsalicyl)-altrosylamine; N,O-di(O-acetylsalicyl)-glucosylamine [same as N,O-di(O-acetylsalicyl)-glucamine]; N,O-di(O-acetylsalicyl)-mannosylamine; N,O-di(O-acetylsalicyl)-gulosylamine; N,O-di(O-acetylsalicyl)-idosylamine; N,O-di(O-acetylsalicyl)-galactosylamine; N,O-di(O-acetylsalicyl)-talosylamine; N,O-di(O-acetylsalicyl)-alloheptosylamine; N,O-di(O-acetylsalicyl)-altroheptosylamine; N,O-di(O-acetylsalicyl)-glucoheptosylamine; N,O-di(O-acetylsalicyl)-mannoheptosylamine; N,O-di(O-acetylsalicyl)-guloheptosylamine; N,O-di(O-acetylsalicyl)-idoheptosylamine; N,O-di(O-acetylsalicyl)-galactoheptosylamine; and N,O-di(O-acetylsalicyl)-taloheptosylamine.

9. The composition of claim 1, wherein the O-acetylsalicyl amino acid has the formula:
R₁CHNHR₂(CH₂)ₙCOR₃,
where R₁ is H, an alkyl, aralkyl or aryl group having 1 to 14 carbon atoms, and R₁ can also carry OH, SH, SCH₃, NH₂, NHR₂, CONH₂, NHCONH₂, NHC(═NH)NH₂, NHC(═NR₂)NH₂ imidazole, pyrrolidine or other heterocyclic group; R₂ is O-acetylsalicyl radical, represented by CH₃COOC₆H₄CO; n is an integer from 0 to 5; R₃ is NH₂, OR₄; R₄ is H, an alkyl, aralkyl or aryl group having 1 to 14 carbon atoms; and the H attached to any carbon atom can be substituted by I, F, Cl, Br, OH or alkoxy group having 1 to 9 carbons.

10. The composition of claim 9, wherein the O-acetylsalicyl amino acid is selected from at least one of the group consisting of N—(O-acetylsalicyl)amino acids: N—(O-acetylsalicyl)-alanine, N—(O-acetylsalicyl)-arginine; N,N′-di(O-acetylsalicyl)-arginine, N—(O-acetylsalicyl)-asparagine, N—(O-acetylsalicyl)-aspartic acid, N—(O-acetylsalicyl)-cysteine, N—(O-acetylsalicyl)-glycine, N—(O-acetylsalicyl)-glutamic acid, N—(O-acetylsalicyl)-glutamine, N—(O-acetylsalicyl)-histidine; N,N′-di(O-acetylsalicyl)-histidine,N—(O-acetylsalicyl)-isoleucine, N—(O-acetylsalicyl)-leucine, N—(O-acetylsalicyl)-lysine; N,N-di(O-acetylsalicyl)-lysine, N—(O-acetylsalicyl)-methionine, N—(O-acetylsalicyl)-phenylalanine, N—(O-acetylsalicyl)-proline, N—(O-acetylsalicyl)-serine, N—(O-acetylsalicyl)-threonine, N—(O-acetylsalicyl)-tryptophan; N,N′-di(O-acetylsalicyl)-tryptophan, N—(O-acetylsalicyl)-tyrosine, N—(O-acetylsalicyl)-valine, N—(O-acetylsalicyl)-β-alanine, N—(O-acetylsalicyl)-γ-aminobutanoic acid, N—(O-acetylsalicyl)-β-aminoisobutanoic acid, N—(O-acetylsalicyl)-citrulline, N—(O-acetylsalicyl)-dopa(3,4-dihydroxyphenylalanine), N—(O-acetylsalicyl)-homocysteine, N—(O-acetylsalicyl)-homoserine, N—(O-acetylsalicyl)-ornithine; N,N-di(O-acetylsalicyl)-ornithine N—(O-acetylsalicyl)-phenylglycine and N—(O-acetylsalicyl)-4-hydroxyphenylglycine.

11. The composition of claim 1, wherein the O-acetylsalicyl peptide has the formula:
R₁CHNHR₂CONHCHR₁COR₃,
where R₁ is independently H, an alkyl, aralkyl or aryl group having 1 to 14 carbon atoms, and can carry OH, SH, SCH₃, NH₂, CONH₂, NHCONH₂, NHC(═NH)NH₂, imidazole, pyrrolidine or other heterocyclic group; R₂ is O-acetylsalicyl radical, represented by CH₃COOC₆H₄CO; R₃ is NH₂, OR₄; R₄ is H, an alkyl, aralkyl or aryl group having 1 to 14 carbon atoms; and the H attached to any carbon atom can be substituted by I, F, Cl, Br, OH or alkoxy group having 1 to 9 carbons.

12. The composition of claim 11, wherein the O-acetylsalicyl peptide is a dipeptide selected from at least one of the group consisting of N—(O-acetylsalicyl)-kyotorphin (Tyr-Arg), N—(O-acetylsalicyl)-anserine (β-Ala-1-N-Me-His), N—(O-acetylsalicyl)-β-Ala-Lys; N—(O-acetylsalicyl)-Asp-Glu, N—(O-acetylsalicyl)-carnosine (β-Ala-His), N—(O-acetylsalicyl)-Gly-Gln, N—(O-acetylsalicyl)-γ-D-Glu-Gly, N—(O-acetylsalicyl)-γ-Glu-Val, N—(O-acetylsalicyl)-γ-Glu-Ile, N—(O-acetylsalicyl)-γ-Glu-Leu, N—(O-acetylsalicyl)-γ-Glu-Met, N—(O-acetylsalicyl)-γ-Glu-Phe, N—(O-acetylsalicyl)-γ-Glu-Ala, N—(O-acetylsalicyl)-γ-Glu-Tyr, N—(O-acetylsalicyl)-γ-Glu-Phe, N—(O-acetylsalicyl)-Leu-Leu, N—(O-acetylsalicyl)-ophidine (β-Ala-2-Me-His), N—(O-acetylsalicyl)-Met-Phe, N—(O-acetylsalicyl)-Ser-Tyr-NH₂, and N—(O-acetylsalicyl)-Tyr-Pro-NH₂.

13. The composition of claim 11, wherein the O-acetylsalicyl peptide is a tripeptide selected from at least one of the group consisting of N—(O-acetylsalicyl)-bursin (Lys-His-Gly-N H₂), N—(O-acetylsalicyl)-D-Phe-Phe-Gly; N—(O-acetylsalicyl)-diprotin A (Ile-Pro-Ile), N—(O-acetylsalicyl)-glutathione (γ-Glu-Cys-Gly), N—(O-acetylsalicyl)-pGlu-Glu-Pro-NH₂, N—(O-acetylsalicyl)-Lys-Trp-Lys, N—(O-acetylsalicyl)-Lys-Tyr-Lys, N—(O-acetylsalicyl)-ophthalmic acid, N—(O-acetylsalicyl)-norophthalmic acid (γ-Glu-Ala-Gly), N—(O-acetylsalicyl)-homoglutathione(bis-γ-Glu-Cys-bis-β-Ala), N—(O-acetylsalicyl)-Met-Leu-Phe, N—(O-acetylsalicyl)-Tyr-Gly-Gly, and N—(O-acetylsalicyl)-Pro-Leu-Gly-NH₂.

14. The composition of claim 11, wherein the O-acetylsalicyl peptide is an oligopeptide selected from at least one of the group consisting of N—(O-acetylsalicyl)-Lys-Lys-Gly-Glu, N—(O-acetylsalicyl)-Arg-Pro-Lys-Pro, N—(O-acetylsalicyl)-Gly-Gly-Phe-Leu, N—(O-acetylsalicyl)-Arg-Gly-Asp-Ser, N—(O-acetylsalicyl)-Arg-Gly-Glu-Ser, N—(O-acetylsalicyl)-Met-Leu-Phe-Phe, N—(O-acetylsalicyl)-Val-Ala-Pro-Gly, N—(O-acetylsalicyl)-Val-Gly-Ser-Glu, N—(O-acetylsalicyl)-Ala-Pro-Gly-Trp-NH₂, N—(O-acetylsalicyl)-Gly-Gly-Phe-Met-NH₂, N—(O-acetylsalicyl)-endorphin 1 (Tyr-Pro-Trp-Phe-NH₂), N—(O-acetylsalicyl)-endorphin 2 (Tyr-Pro-Phe-Phe-NH₂), N—(O-acetylsalicyl)-morphiceptin (Tyr-Pro-Phe-Pro-NH₂), N—(O-acetylsalicyl)-exorphin C (Tyr-Pro-Ile-Ser-Leu), N—(O-acetylsalicyl)-pyroGlu-Glu-Asp-Ser-Gly, N—(O-acetylsalicyl)-Tyr-Gly-Gly-Phe-Leu, N—(O-acetylsalicyl)-Tyr-Gly-Gly-Phe-Met, N—(O-acetylsalicyl)-pyroGlu-Leu-Tyr-Glu-Asn-Lys, N—(O-acetylsalicyl)-Arg-Arg-Pro-Tyr-Ile-Leu, N—(O-acetylsalicyl)-Val-Gly-Val-Ala-Pro-Gly, N—(O-acetylsalicyl)-Tyr-Pro-Ile-Ser-Leu, N—(O-acetylsalicyl)-Arg-Lys-Arg-Ala-Arg-Lys-Glu, N—(O-acetylsalicyl)-kemptide (Leu-Arg-Arg-Ala-Ser-Leu-Gly), N—(O-acetylsalicyl)-Tyr-Gly-Gly-Phe-Leu-Arg-Arg, N—(O-acetylsalicyl)-Met-Glu-His-Phe-Arg-Trp-Gly, N—(O-acetylsalicyl)-Arg-Val-Tyr-Ile-His-Pro-Phe, N—(O-acetylsalicyl)-β-casomorphin (Tyr-Pro-Phe-Val-Glu-Pro-Ile), N—(O-acetylsalicyl)-Asp-Arg-Val-Tyr-Ile-His-Pro-Phe, N—(O-acetylsalicyl)-Met-Gln-Met-Lys-Lys-Val-Leu-Asp-Ser, N—(O-acetylsalicyl)-Arg-Pro-Pro-Gly-Phe-Ser-Pro-Phe-Arg, N—(O-acetylsalicyl)-Ile-Ala-Arg-Arg-His-Pro-Tyr-Phe-Leu, N—(O-acetylsalicyl)-Cys-Tyr-Ile-Gln-Asn-Cys-Pro-Leu-Gly-NH₂, N—(O-acetylsalicyl)-Cys-Tyr-Phe-Gln-Asn-Cys-Pro-Arg-Gly-NH₂, and N—(O-acetylsalicyl)-Cys-Tyr-Ile-Gln-Asn-Cys-Pro-Arg-Gly-NH₂.

15. The composition of claim 1, wherein the O-acetylsalicyl derivative is selected from at least one of the group consisting of O—(O-acetylsalicyl)-amino acids; N,O-di(O-acetylsalicyl)-amino acids; N,O,O-tri(O-acetylsalicyl)-amino acids; S—(O-acetylsalicyl)-amino acids; N,S-di(O-acetylsalicyl)-amino acids; O,S-di(O-acetylsalicyl)-amino acids, and are derived from those amino acids with hydroxyl or thiol group(s). Other (O-acetylsalicyl)-derivatives include N—(O-acetylsalicyl)-amino-oligosaccharides, N—(O-acetylsalicyl)-oligo-aminosaccharides; N,O-di(O-acetylsalicyl)-amino-oligosaccharides and N,O-di(O-acetylsalicyl)-oligo-aminosaccharides.

16. The composition of claim 1, wherein the O-acetylsalicyl derivative is selected from at least one of the group consisting of S—(O-acetylsalicyl)-cysteine; N,S-di(O-acetylsalicyl)-cysteine; S—(O-acetylsalicyl)-homocysteine; N,S-di(O-acetylsalicyl)-homocysteine; O—(O-acetylsalicyl)-4-hydroxyphenylglycine; N,O-di(O-acetylsalicyl)-4-hydroxyphenylglycine; O—(O-acetylsalicyl)-serine; N,O-di(O-acetylsalicyl)-serine; O—(O-acetylsalicyl)-homoserine; N,O-di(O-acetylsalicyl)-homoserine; O—(O-acetylsalicyl)-3-phenylserine; N,O-di(O-acetylsalicyl)-3-phenylserine; O—(O-acetylsalicyl)-threonine; N,O-di(O-acetylsalicyl)-threonine, O—(O-acetylsalicyl)-tyrosine; N,O-di(O-acetylsalicyl)-tyrosine, O—(O-acetylsalicyl)-dopa; O,O-di(O-acetylsalicyl)-dopa; N,O-di(O-acetylsalicyl)-dopa and N,O,O-tri(O-acetylsalicyl)-dopa.

17. The composition of claim 1, wherein the O-acetylsalicyl derivative is N—(O-acetylsalicyl)-L-tyrosine ethyl ester.

18. A method of preventing or treating a disorder or syndrome comprising systemically or topically administering the composition of claim 1, wherein the disorder or syndrome is associated with any one of the nervous, vascular, musculoskeletal or cutaneous systems.

19. The method of claim 18, wherein the O-acetylsalicyl derivative is selected from at least one of the group consisting of N—(O-acetylsalicyl)-glucosamine, N—(O-acetylsalicyl)-mannosamine, N—(O-acetylsalicyl)-galactosamine, N—(O-acetylsalicyl)-glucamine N—(O-acetylsalicyl)-glycine ethyl ester, N—(O-acetylsalicyl)-tyrosine ethyl ester, N—(O-acetylsalicyl)-proline, N—(O-acetylsalicyl)-prolinamide, N—(O-acetylsalicyl)-proline ethyl ester, N—(O-acetylsalicyl)-glutamine, N-(O-acetylsalicyl)-glutamic acid, N—(O-acetylsalicyl)-glutamic acid diethyl ester, N—(O-acetylsalicyl)-arginine, N—(O-acetylsalicyl)-4-hydroxyphenylglycine ethyl ester; N,O-di(O-acetylsalicyl)-4-hydroxyphenylglycine ethyl ester, N—(O-acetylsalicyl)-3-phenylserine; N—(O-acetylsalicyl)-3-phenylserine ethyl ester; N—(O-acetylsalicyl)-lysine and N—(O-acetylsalicyl)-ornithine.

20. The method of claim 18, wherein N—(O-acetylsalicyl)-tyrosine ethyl ester is administered orally to prevent or treat disorders of the musculoskeletal system, selected from the group consisting of osteoporosis, osteoarthritis, rheumatoid arthritis, ankylosing spondylitis, bursitis and tendonitis.

21. The composition of claim 1, further comprising an additional agent selected from at least one of the group consisting of hydroxyacids, ketoacids; phenyl alpha acyloxyalkanoic acids and derivatives; N-acetyl-aldosamines, N-acetylamino acids and related N-acetyl compounds; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotion sickness agents; antiinflammatory agents; antihyperkeratotic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; astringents; cleansing agents; corn, callus and wart removing agents; topical cardiovascular agents; vitamins; corticosteroids; tanning agents; hormones; retinoids; gum disease or oral care agents.

22. The composition of claim 21, wherein said additional agent is selected from at least one of the group consisting of abacavir, acebutolol, acetaminophen, acetaminosalol, acetazolamide, acetohydroxamic acid, N-acetyl-cysteine, N-acetyl-glucosamine, N-acetyl-glutamine, N-acetyl-lysine, N-acetyl-ornithine, N-acetyl-prolinamide, N-acetyl-proline, acetylsalicylic acid, acitretin, aclovate, acrivastine, actiq, acyclovir, adapalene, adefovir dipivoxil, adenosine, albuterol, alfuzosin, allopurinol, alloxanthine, almotriptan, alprazolam, alprenolol, aluminum acetate, aluminum chloride, aluminum chlorohydroxide, aluminum hydroxide, amantadine, amiloride, aminacrine, aminobenzoic acid (PABA), aminocaproic acid, aminosalicylic acid, amiodarone, amitriptyline, amlodipine, amocarzine, amodiaquin, amorolfine, amoxapine, amphetamine, ampicillin, anagrelide, anastrozole, anthralin, apomorphine, aprepitant, arbutin, aripiprazole, ascorbic acid, ascorbyl palmitate, atazanavir, atenolol, atomoxetine, atropine, azathioprine, azelaic acid, azelastine, azithromycin, bacitracin, beclomethasone dipropionate, bemegride, benazepril, bendroflumethiazide, benzocaine, benzonatate, benzophenone, benzoyl peroxide, benztropine, bepridil, betamethasone dipropionate, betamethasone valerate, brimonidine, brompheniramine, bupivacaine, buprenorphine, bupropion, burimamide, butenafine, butoconazole, cabergoline, caffeic acid, caffeine, calcipotriene, camphor, candesartan cilexetil, capsaicin, carbamazepine, carbamide peroxide, cefditoren pivoxil, cefepime, cefpodoxime proxetil, celecoxib, cetirizine, cevimeline, chitosan, chlordiazepoxide, chlorhexidine, chloroquine, chlorothiazide, chloroxylenol, chlorpheniramine, chlorpromazine, chlorpropamide, ciclopirox, cilostazol, cimetidine, cinacalcet, ciprofloxacin, citalopram, citric acid, cladribine, clarithromycin, clemastine, clindamycin, clioquinol, clobetasol propionate, clomiphene, clonidine, clopidogrel, clotrimazole, clozapine, coal tar, cocaine, codeine, cromolyn, crotamiton, cyclizine, cyclobenzaprine, cycloserine, cytarabine, dacarbazine, dalfopristin, dapsone, daptomycin, daunorubicin, deferoxamine, dehydroepiandrosterone, delavirdine, desipramine, desloratadine, desmopressin, desoximetasone, dexamethasone, dexmedetomidine, dexmethylphenidate, dexrazoxane, dextroamphetamine, diazepam, dicyclomine, didanosine, dihydrocodeine, dihydromorphine, diltiazem, 6,8-dimercaptooctanoic acid (dihydrolipoic acid), diphenhydramine, diphenoxylate, dipyridamole, disopyramide, dobutamine, dofetilide, dolasetron, donepezil, dopa esters, dopamide, dopamine, dorzolamide, doxepin, doxorubicin, doxycycline, doxylamine, doxypin, duloxetine, dyclonine, econazole, eflornithine, eletriptan, emtricitabine, enalapril, ephedrine, epinephrine, epinine, epirubicin, eptifibatide, ergotamine, erythromycin, escitalopram, esmolol, esomeprazole, estazolam, estradiol, ethacrynic acid, ethinyl estradiol, etidocaine, etomidate, famciclovir, famotidine, felodipine, fentanyl, ferulic acid, fexofenadine, flecainide, fluconazole, flucytosine, fluocinolone acetonide, fluocinonide, 5-fluorouracil, fluoxetine, fluphenazine, flurazepam, fluvoxamine, formoterol, furosemide, galactarolactone, galactonic acid, galactonolactone, galantamine, gatifloxacin, gefitinib, gemcitabine, gemifloxacin, glucarolactone, gluconic acid, gluconolactone, glucuronic acid, glucuronolactone, glycolic acid, griseofulvin, guaifenesin, guanethidine, N-guanylhistamine, haloperidol, haloprogin, hexylresorcinol, homatropine, homosalate, hydralazine, hydrochlorothiazide, hydrocortisone, hydrocortisone 21-acetate, hydrocortisone 17-butyrate, hydrocortisone 17-valerate, hydrogen peroxide, hydromorphone, hydroquinone, hydroquinone monoether, hydroxyzine, hyoscyamine, hypoxanthine, ibuprofen, ichthammol, idarubicin, imatinib, imipramine, imiquimod, indinavir, indomethacin, irbesartan, irinotecan, isoetharine, isoproterenol, itraconazole, kanamycin, ketamine, ketanserin, ketoconazole, ketoprofen, ketotifen, kojic acid, labetalol, lactic acid, lactobionic acid, lamivudine, lamotrigine, lansoprazole, letrozole, leuprolide, levalbuterol, levofloxacin, lidocaine, linezolid, lobeline, loperamide, losartan, loxapine, lysergic diethylamide, mafenide, malic acid, maltobionic acid, mandelic acid, maprotiline, mebendazole, mecamylamine, meclizine, meclocycline, memantine, menthol, meperidine, mepivacaine, mercaptopurine, mescaline, metanephrine, metaproterenol, metaraminol, metformin, methadone, methamphetamine, methotrexate, methoxamine, methyldopa esters, methyidopamide, 3,4-methylenedioxymethamphetamine, methyllactic acid, methyl nicotinate, methylphenidate, methyl salicylate, metiamide, metolazone, metoprolol, metronidazole, mexiletine, miconazole, midazolam, midodrine, miglustat, minocycline, minoxidil, mirtazapine, mitoxantrone, moexiprilat, molindone, monobenzone, morphine, moxifloxacin, moxonidine, mupirocin, nadolol, naftifine, nalbuphine, nalmefene, naloxone, naproxen, nefazodone, nelfinavir, neomycin, nevirapine, nicardipine, nicotine, nifedipine, nimodipine, nisoldipine, nizatidine, norepinephrine, nystatin, octopamine, octreotide, octyl methoxycinnamate, octyl salicylate, ofloxacin, olanzapine, olmesartan medoxomil, olopatadine, omeprazole, ondansetron, oxiconazole, oxotremorine, oxybenzone, oxybutynin, oxycodone, oxymetazoline, padimate O, palonosetron, pantothenic acid, pantoyl lactone, paroxetine, pemoline, penciclovir, penicillamine, penicillins, pentazocine, pentobarbital, pentostatin, pentoxifylline, pergolide, perindopril, permethrin, phencyclidine, pheneizine, pheniramine, phenmetrazine, phenobarbital, phenol, phenoxybenzamine, phentolamine, phenylephrine, phenylpropanolamine, phenytoin, physostigmine, pilocarpine, pimozide, pindolol, pioglitazone, pipamazine, piperonyl butoxide, pirenzepine, podofilox, podophyllin, povidone iodine, pramipexole, pramoxine, prazosin, prednisone, prenalterol, prilocaine, procainamide, procaine, procarbazine, promazine, promethazine, promethazine propionate, propafenone, propoxyphene, propranolol, propylthiouracil, protriptyline, pseudoephedrine, pyrethrin, pyrilamine, pyrimethamine, quetiapine, quinapril, quinethazone, quinidine, quinupristin, rabeprazole, reserpine, resorcinol, retinal, 13-cis retinoic acid, retinoic acid, retinol, retinyl acetate, retinyl palmitate, ribavirin, ribonic acid, ribonolactone, rifampin, rifapentine, rifaximin, riluzole, rimantadine, risedronic acid, risperidone, ritodrine, rivastigmine, rizatriptan, ropinirole, ropivacaine, salicylamide, salicylic acid, salmeterol, scopolamine, selegiline, selenium sulfide, serotonin, sertindole, sertraline, shale tar, sibutramine, sildenafil, sotalol, streptomycin, strychnine, sulconazole, sulfabenz, sulfabenzamide, sulfabromomethazine, sulfacetamide, sulfachlorpyridazine, sulfacytine, sulfadiazine, sulfadimethoxine, sulfadoxine, sulfaguanole, sulfalene, sulfamethizole, sulfamethoxazole, sulfanilamide, sulfapyrazine, sulfapyridine, sulfasalazine, sulfasomizole, sulfathiazole, sulfisoxazole, sulfur, tadalafil, tamsulosin, tartaric acid, tazarotene, tegaserol, telithromycin, telmisartan, temozolomide, tenofovir disoproxil, terazosin, terbinafine, terbutaline, terconazole, terfenadine, tetracaine, tetracycline, tetrahydrozoline, theobromine, theophylline, thiabendazole, thioctic acid (lipoic acid), thioridazine, thiothixene, thymol, tiagabine, timolol, tinidazole, tioconazole, tirofiban, tizanidine, tobramycin, tocainide, tolazoline, tolbutamide, tolnaftate, tolterodine, tramadol, tranylcypromine, trazodone, triamcinolone acetonide, triamcinolone diacetate, triamcinolone hexacetonide, triamterene, triazolam, triclosan, triflupromazine, trimethoprim, trimipramine, tripelennamine, triprolidine, tromethamine, tropic acid, tyramine, undecylenic acid, urea, urocanic acid, ursodiol, vardenafil, venlafaxine, verapamil, vitamin E acetate, voriconazole, warfarin, wood tar, xanthine, zafirlukast, zaleplon, zinc pyrithione, ziprasidone, zolmitriptan, zolpidem.
